(19) United States
(12) Patent Application Publication
(10) Pub. No.: US 2026/0112469 A1
(43) Pub. Date: Apr. 23, 2026

(54) System and Method for Transformer-Based Network Medicine

(71) Applicant: Northeastern University, Boston, MA (US)

(72) Inventors: Albert-László Barábasi, Brookline, MA (US); Michael L. Sebek, Brookline, MA (US); Andrés Aldana Gonzalez, Boston, MA (US); Jonah Spector, Jamaica Plain, MA (US); Christian de Frondeville, Boston, MA (US); Joseph Ehlert, Boston, MA (US); Abele Sandor Elekes, Boston, MA (US)

(21) Appl. No.: 19/310,706

(22) Filed: Aug. 26, 2025

Related U.S. Application Data
(60) Provisional application No. 63/687,289, filed on Aug. 26, 2024.

(51) Int. Cl. G16H 20/10; G16B 5/00; G16H 50/70

(52) U.S. Cl. CPC G16H 20/10; G16B 5/00; G16H 50/70

(57) ABSTRACT

A computer-based system and computer-implemented method are employed for network medicine. The network medicine employs representations of interactions of biological components. The computer-implemented method employs a machine-readable representation of a transformer weighted protein-protein interactome (PPI) network to perform a computer-informed network medicine task. The machine-readable representation of the transformer weighted PPI network includes machine-readable links weighted based on biological context information extracted from a transformer-based model. The machine-readable links are interposed between machine-readable representations of proteins in the transformer weighted PPI network. The biological context information extracted from the transformer-based model is based on machine-readable genetic data. The computer-implemented method outputs a result of the computer-informed network medicine task performed.

### RELATED APPLICATION

This application claims the benefit of U.S. Provisional Application No. 63/687,289, filed on Aug. 26, 2024. The entire teachings of the above application are incorporated herein by reference.

### BACKGROUND

Network medicine is an interdisciplinary field that applies network theory and systems biology to understand diseases as complex systems of interacting biological components, such as proteins, genes, and metabolites for non-limiting examples.

### SUMMARY

According to an example embodiment, a computer-based system for network medicine comprises at least one processor and a memory. The network medicine employs representations of interactions of biological components. The memory has encoded thereon a sequence of instructions which, when loaded and executed by the at least one processor, causes the computer-based system to employ a machine-readable representation of a transformer weighted protein-protein interactome (PPI) network to perform a computer-informed network medicine task. The machine-readable representation of the transformer weighted PPI network includes machine-readable links weighted based on biological context information extracted from a transformer-based model. The machine-readable links are interposed between machine-readable representations of proteins in the transformer weighted PPI network. The biological context information is extracted from the transformer-based model based on machine-readable genetic data. The sequence of instructions, when loaded and executed by the at least one processor, further causes the computer-based system to output a result of the computer-informed network medicine task performed.

The computer-informed network medicine task may include at least one of: disease gene discovery and drug repurposing for non-limiting examples.

The machine-readable genetic data may represent genetic data of cells of an individual with a disease or genetic data of cells of a plurality individuals associated with the disease for non-limiting examples.

The computer-informed network medicine task performed may include predicting, from an input set of candidate drugs, which candidate drugs of the input set are useful to treat a disease. The result output may be a prioritized list of candidate drugs from the input set of candidate drugs predicted to treat the disease. A subset of the input set of candidate drugs may be known to treat the disease. A total number of candidate drugs in the prioritized list of candidate drugs may be reduced relative to a total number of candidate drugs in the input set.

The computer-informed network medicine task performed may include predicting from an input set of candidate drugs known to treat a disease and a machine-readable genetic profile of an individual with the disease, which candidate drugs of the input set are useful to treat the disease in the individual. The result output may be a prioritized list of candidate drugs from the input set predicted to treat the disease. A location within the prioritized list may represent probability of success in treating the disease.

The computer-informed network medicine task performed may include predicting from an input set of candidate drugs and a machine-readable genetic profile of an individual with a disease, which candidate drugs of the input set are useful to treat the disease in the individual. The result output may be a prioritized list of candidate drugs from the input set predicted to treat the disease.

The computer-informed network medicine task performed may include discovering new genes associated with a disease based on known genes associated with the disease. The result output may be a prioritized list of new genes determined to be associated with the disease.

The computer-informed network medicine task performed may include determining which drugs of an input set of candidate drugs with respective disease targets can be used to treat a disease different from the respective disease targets. The result output may be a prioritized list of the input set.

The sequence of instructions, when loaded and executed by the at least one processor, may further cause the computer-based system to transform a machine-readable representation of an unweighted PPI into the transformer weighted PPI network by determining weights based on the biological context information extracted from the transformer-based model and assigning the weights determined to machine-readable links interposed between machine-readable representations of proteins in the unweighted PPI network.

The biological context information extracted from the transformer-based model may include machine-readable vectors of different layers of the transformer-based model that represent respective expression levels of genes in cells represented in the machine-readable genetic data. The sequence of instructions, when loaded and executed by the at least one processor, may further cause the computer-based system to compute angles between the machine-readable vectors of the different layers. The machine-readable links may be weighted based on the angles computed.

The sequence of instructions, when loaded and executed by the at least one processor, may further cause the computer-based system to aggregate the angles computed, compute an average value of the angle computed and aggregated, and assign weights to the machine-readable links based on the average value computed.

The biological context information extracted from the transformer-based model may include attention weights extracted from the transformer-based model. The sequence of instructions, when loaded and executed by the at least one processor, may further cause the computer-based system to assign weights to the machine-readable links based on the attention weights extracted.

According to another example embodiment, a computer-implemented method for network medicine, the network medicine employing representations of interactions of biological components, comprises employing a machine-readable representation of a transformer weighted protein-protein interactome (PPI) network to perform a computer-informed network medicine task. The machine-readable representation of the transformer weighted PPI network includes machine-readable links weighted based on biological context information extracted from a transformer-based model. The machine-readable links are interposed between machine-readable representations of proteins in the transformer weighted PPI network. The biological context information extracted from the transformer-based model is based on machine-readable genetic data. The computer-implemented method further comprises outputting a result of the computer-informed network medicine task performed.

Further alternative computer-implemented method embodiments parallel those described above in connection with the example computer-based system embodiment.

According to another example embodiment, a non-transitory computer-readable medium for network medicine has encoded thereon a sequence of instructions. The network medicine employs representations of interactions of biological components. The sequence of instructions, when loaded and executed by at least one processor, causes the at least one processor to employ a machine-readable representation of a transformer weighted protein-protein interactome (PPI) network to perform a computer-informed network medicine task. The machine-readable representation of the transformer weighted PPI network includes machine-readable links weighted based on biological context information extracted from a transformer-based model. The machine-readable links are interposed between machine-readable representations of proteins in the transformer weighted PPI network. The biological context information is extracted from the transformer-based model based on machine-readable genetic data. The sequence of instructions, when loaded and executed by at least one processor, further causes the at least one processor to output a result of the computer-informed network medicine task performed.

Further alternative non-transitory computer-readable medium embodiments parallel those described above in connection with the example computer-based system embodiment.

It should be understood that example embodiments disclosed herein can be implemented in the form of a method, apparatus, system, or computer readable medium with program codes embodied thereon.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a block diagram of an example embodiment of a computer-based system for network medicine.

FIG. 2 is a block diagram of an example embodiment of the computer-based system of FIG. 1.

FIG. 3 is a flow diagram of an example embodiment of a computer-implemented method for network medicine.

FIG. 4A is a flow diagram of an example embodiment or a method for training Geneformer.

FIG. 4B is a continuation of FIG. 4A.

FIGS. 5A-G are diagrams that illustrate example embodiments of similarity measurements in Geneformer.

FIG. 6 is a schematic diagram of an example embodiment of a network.

FIG. 7A is a graph of an example embodiment of density vs. attention weight.

FIG. 7B is a graph of an example embodiment of density versus cosine similarity.

FIG. 7C is a graph of an example embodiment of a fraction of edges recovered versus rank

FIG. 8A is a block diagram of an example embodiment of Geneformer used both with additional fine-tuning and without.

FIG. 8B is an illustration of an example embodiment of a disease module in a network.

FIG. 8C is a table of an example embodiment of distributions of attention weights from the pretrained model and the cardiomyopathy model plotted for PPI edges, non-physical connections between disease genes, and disease module edges.

FIG. 8D is a table of an example embodiment of attention weights for both models that are plotted for several different disease modules.

FIG. 9A is a diagram of an example embodiment of a disease module shaded to show different groups of nodes based on how many hops they are away from the disease module.

FIG. 9B is a graph of an example embodiment of attention weight versus hops away from the disease module.

FIG. 10 is a graph of an example embodiment of a fraction of genes recovered per node.

FIG. 11A is a graph of an example embodiment of receiver operating characteristic (ROC) curves.

FIG. 11B is a graph of an example embodiment of cumulative true positives.

FIG. 11C is a graph of an example embodiment of ROC curves for a Geneformer weighted PPI network and a PPI network weighted using a pre-trained Geneformer model.

FIG. 11D is a graph of an example embodiment of cumulative true positives among the top 50 drug candidates for the Geneformer weighted PPI network and the pre-trained Geneformer model weighted PPI network.

FIG. 12 is a graph of an example embodiment of area under curve (AUC) values for benchmark numbers of training samples for different models.

FIG. 13A is a block diagram of an example embodiment of an attention weight comparison method.

FIG. 13B is a graph of an example embodiment of comparison weights.

FIG. 14 is a flow diagram of an example embodiment of a single iteration of a Shapley approximation.

FIG. 15A is a plot of an example embodiment of Shapley values per number of iterations for different genes.

FIG. 15B is a plot of an example embodiment of the Shapley value of genes at different positions in a rank value encoding.

FIG. 15C is a plot of an example embodiment of the Shapley value for 10 genes that are over expressed in disease samples, and 10 genes that occur at the same position as the differentially expressed genes (DEGs), but in random samples.

FIG. 15D is a plot of an example distribution of transcriptome lengths in Geneformer's pretraining corpus.

FIGS. 16A and 16B are example embodiments of heat maps for an attention-based comorbidity analysis for cardiomyopathy.

FIG. 16C is a table of name and comorbidity status of four disease-disease interactions ranked by direct attention weight.

FIGS. 17A and 17B are heat maps of example embodiments of comorbidity comparisons.

FIGS. 18A-C are plots of example embodiments of a relationship between dataset size and model performance for bulk ribonucleic acid (RNA) data and single-cell RNA data with Geneformer.

FIGS. 19A-F are graphs of example embodiments of results of a network-based analysis of non-coding human interactome (NCI) Geneformer attention weights.

FIG. 20 is a block diagram of an example of the internal structure of a computer in which various embodiments of the present disclosure may be implemented.

### DETAILED DESCRIPTION

A description of example embodiments follows.

It should be understood that a transformer-based model disclosed herein is not limited to Geneformer. Further, while a disease disclosed herein may be described as cardiomyopathy, it should be understood that a disease disclosed herein is not limited to cardiomyopathy and may be any other disease or condition.

Transformer models have been applied to biological data by tokenizing single cell ribonucleic acid (RNA) sequencing data just as a large language model (LLM) would tokenize a paragraph of text. These models have achieved unprecedented accuracy on an array of classification tasks, but the inherent abstraction of the gene-based models makes them difficult to interpret. According to an example embodiment disclosed herein, a LLM can be used effectively with bulk RNA data, suggesting that the models may learn some biological information that transcends the single cell level. An example embodiment of a framework disclosed herein may be employed for interpreting one such model. As disclosed herein, the model learns the physical gene-gene interaction network during pretraining, which can then be modified during fine-tuning to assist with different classification tasks.

Network medicine emerged as a paradigm-shifting approach (A.-L. Barabási, N. Gulbahce, and J. Loscalzo. Network medicine: a network-based approach to human disease. Nat Rev Genet, 12(1):56-68, 2011), offering a framework that marries the macroscopic and microscopic perspectives by conceptualizing biological processes as networks of interacting elements at different scales, such as genes, proteins, tissues and organs. Network medicine provides a promising avenue for understanding the systemic nature of health and illness by studying diseases not just as molecular defects of isolated entities but as perturbations in the interactions that connect various cellular components. Network medicine approaches have already made strides in areas like drug re-purposing by identifying promising drugs for the treatment of COVID-19 (Deisy Morselli Gysi, Ítalo do Valle, Marinka Zitnik, Asher Ameli, Xiao Gan, Onur Varol, Susan Dina Ghiassian, J. J. Patten, Robert A. Davey, Joseph Loscalzo, and Albert-László Barabási. Network medicine framework for identifying drug-repurposing opportunities for covid-19. Proceedings of the National Academy of Sciences, 118(19):e2025581118, 2021) and have led to clinical advances in predicting drug responses for rheumatoid arthritis patients. However, the field is currently impeded by significant challenges, including the pervasive noise and complexity inherent in biological data, which complicate the mapping and analysis of the networks. As such, innovative solutions capable of deciphering the complex web of biological interactions are useful, thereby enhancing the accuracy and applicability of network medicine in clinical and research settings. As the understanding of network topology and dynamics remains incomplete, there is a pressing demand for advanced methodologies to handle the multifaceted nature of biological data and provide deeper, more actionable insights into human biology.

Machine learning is one such methodology. By applying complex models with large numbers of trainable parameters, huge strides have been made in demystifying problems, such as drug-target binding (Hakime Öztürk, Arzucan Özgür, and Elif Ozkirimli. DeepDTA: deep drug-target binding affinity prediction. Bioinformatics, 34(17):1821-1829, 09 2018) and identifying genetic variants (Aaron McKenna, Matthew Hanna, Eric Banks, Andrey Sivachenko, Kristian Cibulskis, Andrew Kernytsky, Kiran Garimella, David Altshuler, Stacey Gabriel, Mark Daly, and Mark DePristo. The genome analysis toolkit: A mapreduce framework for analyzing next-generation dna sequencing data. Genome research, 20:1297-303, 09 2010). These approaches, while powerful, are subject to many of the same challenges as network medicine. The complexity of biological data, especially in exploratory settings, often prevents the assembly of the large, labeled datasets needed to train traditional machine learning models. Models that perform well in some settings may be no better than random if their training data is sufficiently limited.

Transformers (Ashish Vaswani, Noam Shazeer, Niki Parmar, Jakob Uszkoreit, Llion Jones, Aidan N Gomez, Lukasz Kaiser, and Illia Polosukhin. Attention is all you need. arXiv, 2017), a class of deep learning models initially designed for natural language processing (NLP), have revolutionized various fields of study, including biology. These models excel at capturing complex patterns and dependencies in data through their self-attention mechanisms, allowing for the analysis and prediction of intricate biological phenomena. In the realm of biology, transformers are applied to tasks ranging from protein structure prediction (Yunha Hwang, Andre L Cornman, Elizabeth H Kellogg, Sergey Ovchinnikov, and Peter R Girguis. Genomic language model predicts protein co-regulation and function. Nature Communications, 15:2880, 2024), understanding genetic sequences (Yanrong Ji, Zhihan Zhou, Han Liu, and Ramana V Davuluri. DNABERT: pre-trained Bidirectional Encoder Representations from Transformers model for DNA-language in genome. Bioinformatics, 37(15):2112-2120, 02 202, (Carlos Outeiral and Charlotte M Deane. Codon language embeddings provide strong signals for use in protein engineering. Nature Machine Intelligence, 6:170-179, 2024), and molecular interactions (Chiranjib Chakraborty, Manojit Bhattacharya, and Sang-Soo Lee. Artificial intelligence enabled chatgpt and large language models in drug target discovery, drug discovery, and development. Molecular Therapy-Nucleic Acids, 33:866-868, 9 2023). A key to their success lies in the pretraining phase, where they learn general features from large datasets, followed by fine-tuning on more specific tasks with limited data. This two-step process enables transformers to transfer knowledge from broad contexts to particular biological challenges, making them exceptionally powerful tools for advancing research and discovery in the life sciences.

An example of a transformer applied to biology is Geneformer (C. V. Theodoris et al. Transfer learning enables predictions in network biology. Nature, 618(7965):616-624, June 2023), a deep learning system, pretrained on a vast corpus comprising approximately 30 million single-cell transcriptomes. Geneformer takes as input the transcriptome of individual cells and can predict transcription factors sensitive to gene dosage, differentiate between methylated and non-methylated genes, and identify cardiomyocytes affected by hypertrophic or dilated cardiomyopathies.

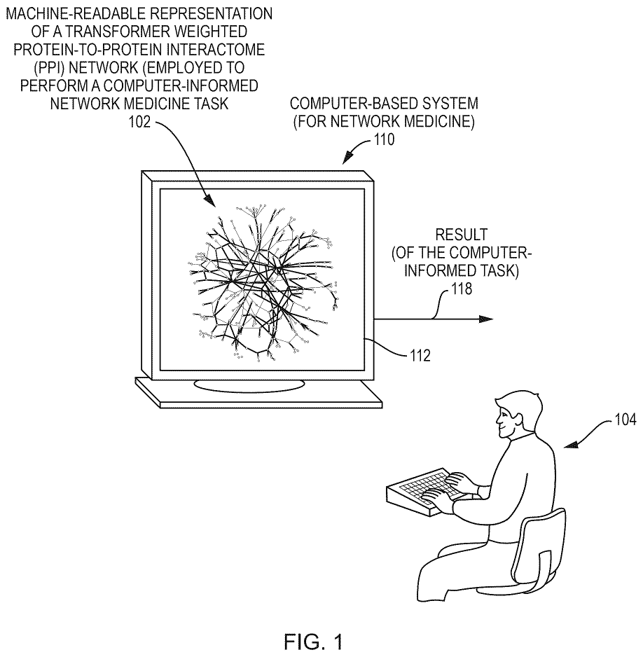

While Geneformer and other similar transformer-based implementations have successfully applied transfer learning to a variety of difficult tasks (H. Cui, C. Wang, H. Maan, and B. Wan. scgpt: Towards building a foundation model for single-cell multi-omics using generative ai. Nature Methods, 2024), (Yiqun T. Chen and James Zou. Genept: A simple but hard-to-beat foundation model for genes and cells built from chatgpt. bioRxiv, 2023), it is not yet known whether the internal elements of the fine-tuned system, such as the embeddings or the attention heads, can contain important information that aids in understanding the phenomena they are predicting. As disclosed herein, the core of Geneformer and biological LLMs more generally, can capture Protein-Protein Interaction (PPI) networks and disease modules of network medicine within the internal attention mechanisms of the pretrained and fine-tuned models, showing that these LLMs do contain relevant biological information. Furthermore, as disclosed herein, LLMs can improve network medicine tasks, such as disease gene discovery and drug repurposing. Overall, research disclosed herein reflects a convergence of disciplines aimed at unraveling the complexities of biological systems at an unprecedented scale and depth, providing a more detailed, dynamic, and predictive model of biological interactions and disease mechanisms. An example embodiment of a computer-based system that may employ such a model is disclosed below with reference to FIG. 1.

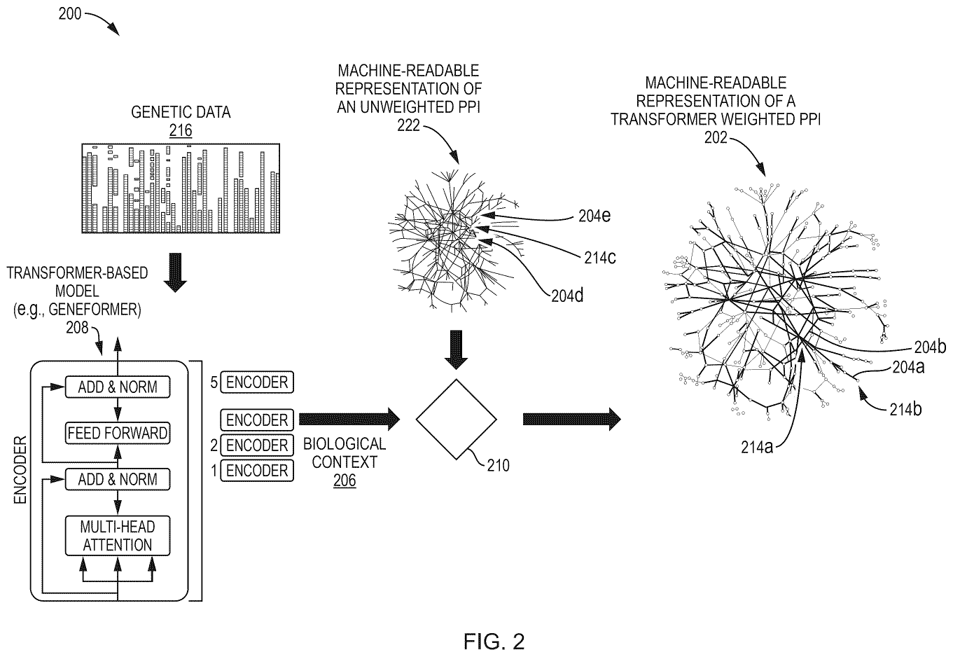

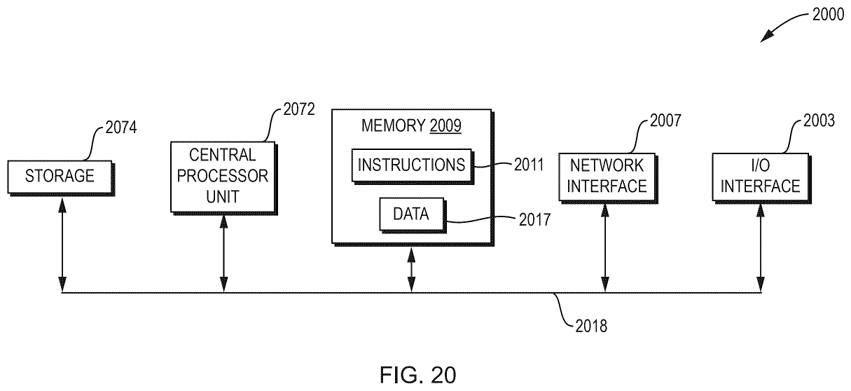

FIG. 1 is a block diagram 100 of an example embodiment of a computer-based system 110 for network medicine. The network medicine may employ representations of interactions of biological components, such as included in a machine-readable representation of a transformer weighted protein-protein interactome (PPI) network 102 visualized on a display device 112 of the computer-based system 110 viewed by a user 104. The computer-based system 110 may comprise at least one processor (not shown) and a memory (not shown), such as disclosed further below in reference to FIG. 20 for non-limiting example. Continuing with reference to FIG. 1, the memory has encoded thereon a sequence of instructions (not shown) which, when loaded and executed by the at least one processor, may cause the computer-based system 110 to employ the machine-readable representation of the transformer weighted PPI network 102 to perform a computer-informed network medicine task. The machine-readable representation of the transformer weighted PPI network 102 includes machine-readable links weighted based on biological context information extracted from a transformer-based model, such as disclosed below with reference to FIG. 2.

FIG. 2 is a block diagram 200 of an example embodiment of the computer-based system 110 of FIG. 1. With reference to FIG. 1 and FIG. 2, the block diagram 200 includes a computer-based system 210 that may be the computer-based system 110, disclosed above. According to an example embodiment, the computer-based system (110, 210) may be configured to generate a machine-readable representation of a transformer weighted PPI network 202, that may be the machine-readable representation of the transformer weighted PPI network 102, disclosed above. Continuing with reference to FIG. 1 and FIG. 2, the machine-readable representation of the transformer weighted PPI network (102, 202) may include machine-readable links (e.g., 204a, 204b, etc.) that may be weighted based on biological context information 206 extracted from a transformer-based model 208, such as geneformer for non-limiting example. The machine-readable links may be interposed between machine-readable representations of proteins (e.g., 214a, 214b, etc.) in the transformer weighted PPI network 202. The biological context information 206 may be extracted from the transformer-based model 208 based on machine-readable genetic data 216. The sequence of instructions, when loaded and executed by the at least one processor, may further cause the computer-based system (110, 210) to output a result 118 of the computer-informed network medicine task performed.

The computer-informed network medicine task may include at least one of: disease gene discovery and drug repurposing for non-limiting examples. The machine-readable genetic data 216 may represent genetic data of cells of an individual (not shown) with a disease or genetic data of cells of a plurality individuals (not shown) associated with the disease for non-limiting examples.

The computer-informed network medicine task performed may include predicting, from an input set of candidate drugs (not shown), which candidate drugs of the input set are useful to treat a disease. The result output 112 may be a prioritized list of candidate drugs (not shown) from the input set of candidate drugs predicted to treat the disease. A subset of the input set of candidate drugs may be known to treat the disease. A total number of candidate drugs in the prioritized list of candidate drugs may be reduced relative to a total number of candidate drugs in the input set.

The computer-informed network medicine task performed may include predicting from an input set of candidate drugs known to treat a disease (not shown) and a machine-readable genetic profile (not shown) of an individual (not shown) with the disease, which candidate drugs of the input set are useful to treat the disease in the individual. The result output 118 may be a prioritized list of candidate drugs (not shown) from the input set predicted to treat the disease. A location (not shown) within the prioritized list may represent probability of success in treating the disease.

The computer-informed network medicine task performed may include predicting from an input set of candidate drugs and a machine-readable genetic profile of an individual with a disease, which candidate drugs of the input set are useful to treat the disease in the individual. The result output 118 may be a prioritized list of candidate drugs from the input set predicted to treat the disease.

The computer-informed network medicine task performed may include discovering new genes (not shown) associated with a disease based on known genes associated with the disease. The result output 118 may be a prioritized list of new genes (not shown) determined to be associated with the disease.

The computer-informed network medicine task performed may include determining which drugs of an input set of candidate drugs with respective disease targets (not shown) can be used to treat a disease different from the respective disease targets. The result output 118 may be a prioritized list of the input set (not shown).

The sequence of instructions, when loaded and executed by the at least one processor, may further cause the computer-based system (110, 210) to transform a machine-readable representation of an unweighted PPI 222 into the transformer weighted PPI network (102, 202) by determining weights (not shown) based on the biological context information 206 extracted from the transformer-based model 208 and assigning the weights determined to machine-readable links interposed between machine-readable representations of proteins in the unweighted PPI network 222, such as the machine-readable link 214c interposed between the machine-readable representations of proteins 204d and 204e for non-limiting example.

The biological context information 206 extracted from the transformer-based model 208 may include machine-readable vectors of different layers of the transformer-based model 208 that represent respective expression levels of genes in cells represented in the machine-readable genetic data 216, as disclosed further below. The sequence of instructions, when loaded and executed by the at least one processor, may further cause the computer-based system (110, 210) to compute angles between the machine-readable vectors of the different layers, as disclosed further below. The machine-readable links may be weighted based on the angles computed.

The sequence of instructions, when loaded and executed by the at least one processor, may further cause the computer-based system (110, 210) to aggregate the angles computed, compute an average value of the angle computed and aggregated, and assign weights to the machine-readable links based on the average value computed, as disclosed further below.

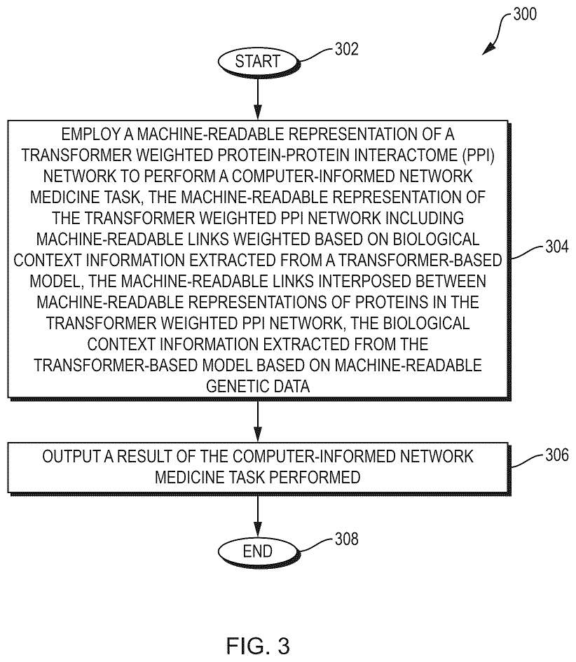

The biological context information 206 extracted from the transformer-based model 208 may include attention weights extracted from the transformer-based model 208, as disclosed further below. The sequence of instructions, when loaded and executed by the at least one processor, may further cause the computer-based system (110, 210) to assign weights to the machine-readable links based on the attention weights extracted, as disclosed further below. An example embodiment of a computer-implemented method for network medicine that may be implemented by the computer-based system 110 to assign such weights is disclosed below in reference to FIG. 3.

FIG. 3 is a flow diagram of an example embodiment of a computer-implemented method for network medicine, the network medicine employing representations of interactions of biological components (300). The computer-implemented method may begin (302) and comprise employing a machine-readable representation of a transformer weighted protein-protein interactome (PPI) network to perform a computer-informed network medicine task (304). The machine-readable representation of the transformer weighted PPI network may include machine-readable links weighted based on biological context information extracted from a transformer-based model. The machine-readable links may be interposed between machine-readable representations of proteins in the transformer weighted PPI network. The biological context information extracted from the transformer-based model may be based on machine-readable genetic data. The computer-implemented method may further comprise outputting a result of the computer-informed network medicine task performed (306). The computer-implemented method thereafter ends (308) in the example embodiment. Further alternative computer-implemented method embodiments parallel those described above in connection with the example embodiment of the computer-based system 110 of FIG. 1.

Further technical details are disclosed below.

### Geneformer and Large Language Models (LLMs) in Single Cell Biology

Transformer models, introduced in 2017 (Ashish Vaswani, Noam Shazeer, Niki Parmar, Jakob Uszkoreit, Llion Jones, Aidan N Gomez, Lukasz Kaiser, and Illia Polosukhin. Attention is all you need. arXiv, 2017) in the field of natural language processing (NLP), are now widely used in many different fields, such as computer vision (Sonain Jamil, Md Jalil Piran, and Oh-Jin Kwon. A comprehensive survey of transformers for computer vision. Drones, 7(5):287, 2023), medical sciences (Hong-Yu Zhou, Yizhou Yu, Chengdi Wang, Shu Zhang, Yuanxu Gao, Jia Pan, Jun Shao, Guangming Lu, Kang Zhang, and Weimin Li. A transformer-based representation-learning model with unified processing of multimodal input for clinical diagnostics. Nature Biomedical Engineering, 7(6):743-755, 2023), climate modeling (Zied Ben Bouallègue, Jonathan A Weyn, Mariana C A Clare, Jesper Dramsch, Peter Dueben, and Matthew Chantry. Improving medium-range ensemble weather forecasts with hierarchical ensemble transformers. Artificial Intelligence for the Earth Systems, 3(1):e230027, 2024) and biology (Abel Chandra, Laura Tünnermann, Tommy Löfstedt, and Regina Gratz. Transformer-based deep learning for predicting protein properties in the life sciences. Elife, 12:082819, 2023), (Sanghyuk Roy Choi and Minhyeok Lee. Transformer architecture and attention mechanisms in genome data analysis: a comprehensive review. Biology, 12(7):1033, 2023). Their application to the complex sequences inherent in biological data (Yanrong Ji, Zhihan Zhou, Han Liu, and Ramana V Davuluri. DNABERT: pre-trained Bidirectional Encoder Representations from Transformers model for DNA-language in genome. Bioinformatics, 37(15):2112-2120, 02 2021), (H. Cui, C. Wang, H. Maan, and B. Wan. scgpt: Towards building a foundation model for single-cell multi-omics using generative ai. Nature Methods, 2024), (C. V. Theodoris et al. Transfer learning enables predictions in network biology. Nature, 618(7965):616-624, June 2023) and represents a new frontier for both biology and Artificial Intelligence (AI) and invites a review of the mechanisms that separate transformers from other models. Geneformer is a deep learning model that leverages the power of self-attention to provide context-sensitive predictions in network biology. Pretrained on 30 million single cell transcriptomes, it employs transfer learning to make accurate predictions in biology. By utilizing self-attention mechanisms, Geneformer identifies and learns which genes to prioritize for enhanced predictive performance. Importantly, Geneformer's context-aware architecture adapts to varying cell dynamics-such as differences across cell types, developmental stages, or disease conditions. This capability ensures that its predictions are finely tuned to the specific characteristics of each cell's context, offering a tailored approach to understanding biological processes.

In NLP, models are given inputs in the form of sequences, which are paragraphs. They are composed of tokens, which are vector representations of the words in the sentence. In biology, a plethora of different types of data have been interpreted as sequences, such as DNA sequences (Yanrong Ji, Zhihan Zhou, Han Liu, and Ramana V Davuluri. DNABERT: pre-trained Bidirectional Encoder Representations from Transformers model for DNA-language in genome. Bioinformatics, 37(15):2112-2120, 02 2021), (Carlos Outeiral and Charlotte M Deane. Codon language embeddings provide strong signals for use in protein engineering. Nature Machine Intelligence, 6:170-179, 2024), transcriptomes (C. V. Theodoris et al. Transfer learning enables predictions in network biology. Nature, 618(7965):616-624, June 2023), (H. Cui, C. Wang, H. Maan, and B. Wan. scgpt: Towards building a foundation model for single-cell multi-omics using generative ai. Nature Methods, 2024), or proteome (Yunha Hwang, Andre L Cornman, Elizabeth H Kellogg, Sergey Ovchinnikov, and Peter R Girguis. Genomic language model predicts protein co-regulation and function. Nature Communications, 15:2880, 2024). A focus herein may be on a transcriptome-based LLM, where the individual tokens are genes. In language, the order of the tokens is predefined by the grammar. In transcriptomics, grammar may be employed to order the tokens.

Geneformer (C. V. Theodoris et al. Transfer learning enables predictions in network biology. Nature, 618(7965):616-624, June 2023) resolves this using Rank-Value Encoding (RVE). Given a list of genes and their expression in a cell, the expression data is first normalized by the median expression of the gene in the pertaining corpus. This deprioritizes housekeeping genes (which are always highly expressed) while preserving the effect of other highly expressed genes. The genes are then ordered by normalized expression to create a ranked list (C. V. Theodoris et al. Transfer learning enables predictions in network biology. Nature, 618(7965):616-624, June 2023), serving as the ‘sentence’ and the input to the model, defining the ‘grammar’ of the cell.

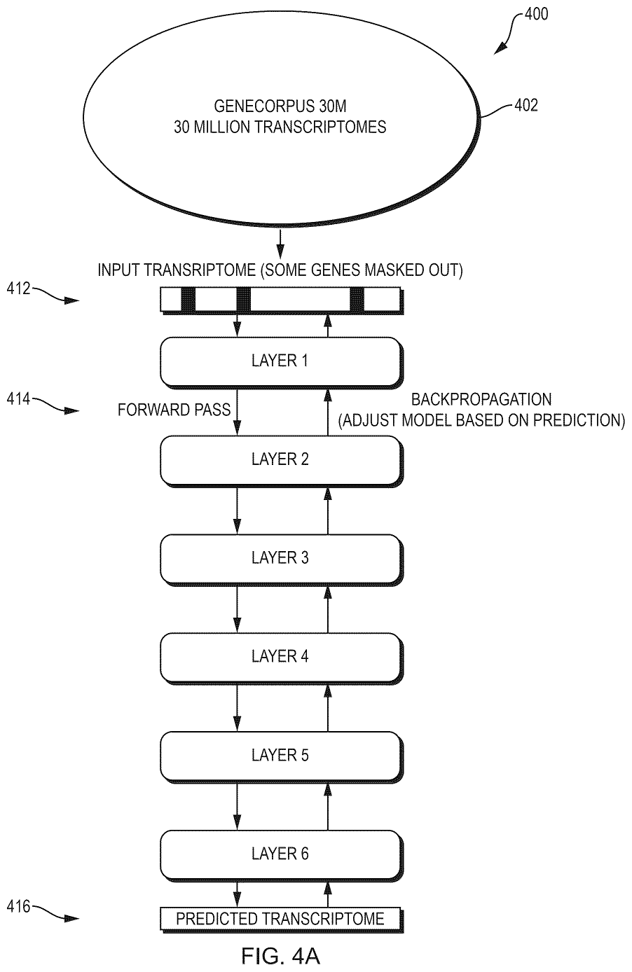

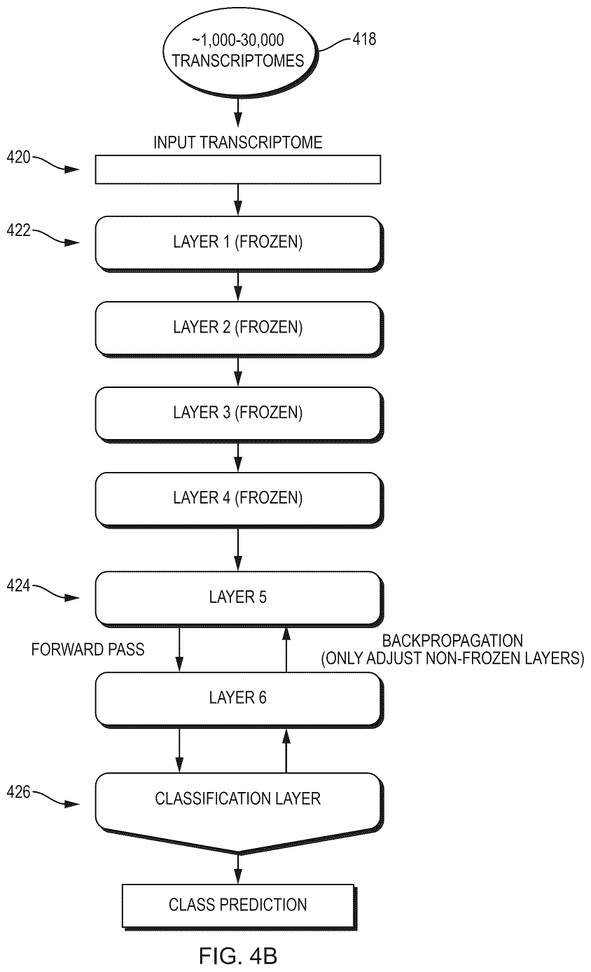

FIG. 4A is a flow diagram 400 of an example embodiment or a method for training Geneformer.

FIG. 4B is a continuation of FIG. 4A. With reference to FIG. 4A and FIG. 4B, at 402, a corpus of 30 million single cell transcriptomes is used for pretraining the model, for non-limiting example. At 412, a sample is selected to be passed through the model, and some genes are masked out. At 414, the sample with masked genes is passed through the model. At 416, the output of the model is a transcriptome where the masked out genes have been predicted by the model. This is compared to the ground truth, and the error is backpropagated through the model to update it. This process is repeated until Geneformer has made predictions for each cell in the corpus (one epoch). At 418, starting with this pretrained model, as well as a smaller corpus of transcriptomes (typically on the order of 10,000) the model can be fine-tuned. At 420, an input transcriptome with a label (for example disease vs. healthy) is passed through the model. At 422, some layers will be frozen, meaning their weights will not be updated during fine-tuning. The number of frozen layers is task specific, ranging from 0-6. The freezing of a layer does not change the forward pass through the model, only the backpropagation. In this case, at 424, layers 5 and 6 remain unfrozen and can be updated. At 426, an additional classification layer is added. This is a transformer encoder layer like all the others, except that instead of outputting a transcriptome, it collapses the output to an array of probabilities for each class. If the model is predicting disease vs healthy, the output would be [p_{healthy}, p_{disease}]. These probabilities are compared to the ground truth label, and the error is backpropagated only through the non-frozen layers. This process takes much less time and resources than fine-tuning and must be done for each new prediction task.

As such, according to the non-limiting example embodiment, Geneformer was pretrained using Genecorpus 30M, which contains 30 million publicly available single cell transcriptomes (see 402). Starting with a randomly initialized model, sequences are input to the model with certain tokens masked out. The model is asked to predict the masked token, and update its weights based on the result. This phase of training gives Geneformer a versatile context awareness that can be harnessed through posterior task-specific training phases (fine-tuning). While pretraining gives the model a general base of knowledge for the context in which ranked genes are found, fine-tuning allows the model to specifically apply these context-dependent relationships to a specific classification problem (see 412). In biology, fine-tuning is particularly useful because it can be performed with relatively small datasets. On certain tasks such as the labeling of gene dosage sensitivity, data can be limited to as few as 10,000 cells. In this scenario, Geneformer outperforms the predictive capabilities of traditional machine learning techniques. While models like support vector machines (SVM), random forest (RF), and feed-forward networks (FFN) are only able to achieve AUCs of 0.70-0.75, the fine-tuned Geneformer achieves an AUC of 0.91 (C. V. Theodoris et al. Transfer learning enables predictions in network biology. Nature, 618(7965):616-624, June 2023). Additionally, Geneformer outpaces its competitors by similar margins when trained to annotate cell types, identify bivalently marked genes, and identify central genes in the regulatory network for cardiac valve disease.

GeneFormer's context awareness comes from its self-attention mechanism, a four-head attention unit that is found in each of its six layers. When a sample is passed through the model, each of its 24 total attention heads creates an n×n attention matrix, where n is the number of genes in the sample. The attention matrices are row normalized and asymmetrical. This means that each gene has the same total attention distributed across the rest of the genes in the sample, and that the attention aᵢⱼ given from gene i to gene j will not generally be the same as the attention aⱼᵢ received by gene i from gene j. These attention heads have different distributions of weights, and often pay attention to different types of genes. The creators of the model observe that several attention heads attend heavily to transcription factors, which play a key role in determining the function of a cell (C. V. Theodoris et al. Transfer learning enables predictions in network biology. Nature, 618(7965):616-624, June 2023).

Given the success of LLMs and Geneformer in particular, an example embodiment may harness its context-aware internal representation and predictive power to enhance tasks in network medicine. These tasks include protein-protein interaction (PPI) detection, identification of disease modules, and discovery of genes associated with diseases for non-limiting examples. An example embodiment provides a more comprehensive framework for understanding complex diseases and exploring potential treatments.

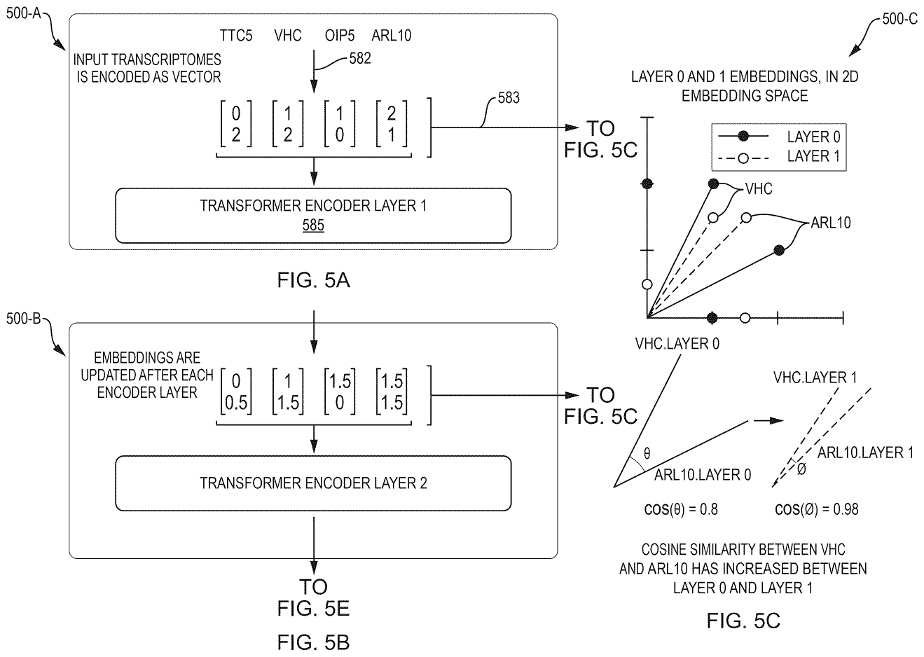

FIGS. 5A-G are diagrams (500-A, 500-B, 500-C, 500-D, 500-E, 500-F, and 500-G) that illustrate example embodiments of similarity measurements in Geneformer. In FIG. 5A, starting with a transcriptome 582, genes are tokenized into vector embeddings 583 and fed through the first transformer encoder layer 585. In FIG. 5B, each layer outputs an updated set of embeddings before passing them into the next layer. In FIG. 5C, the initial embeddings (layer 0) and the layer 1 embeddings are plotted in the plot 500-C to show how the genes can move in embedding space between layers. In this case, VHC and ARL10 get closer in embedding space after layer 1. In FIG. 5D, the layer 0 embeddings are turned into a cosine similarity network 500-D, where each gene is connected to every other gene with an edge weighted by its cosine similarity. Here, ARL10 591 and OIP5 593 are orthogonal in embedding space, so no edge exists. In FIG. 5E, the final layer 587 of the model is expanded to show the multi-head attention mechanism 588. All six layers have an identical mechanism, giving the model 24 total attention heads. In FIG. 5F, an attention head from the 6th layer, that is, the final layer 587, is magnified. These attention weights are another way to compare gene similarities, as each gene attends to every other gene in the sequence. Since the matrix is asymmetric, the attention to TTC5 from ARL10 is different from the attention to ARL10 from TTC5. In FIG. 5G, the layer 6 attention weights are turned into a weighted directed network 500-G. Since the matrix is asymmetrical, there is an edge in both directions for each pair of genes.

### Mapping Geneformer to the PPI

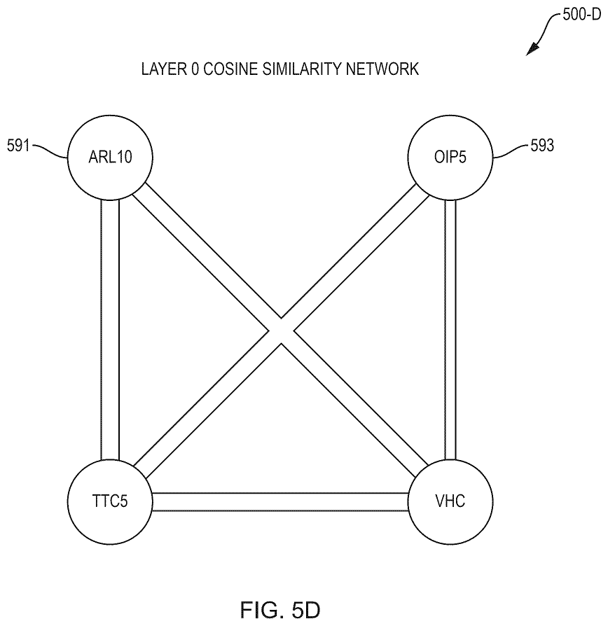

FIG. 6 is a schematic diagram of an example embodiment of a network 600. Calculating the similarity between pairs of genes in Geneformer, whether by attention weights or embedding similarities, produces a fully connected, weighted network 600. Some of this network will not be included in the PPI 608 and is shown in grey. The PPI 608 will be a subset of this fully connected network 600 and is shown in green (bold).

### Results

### GeneFormer Prioritizes PPI Edges

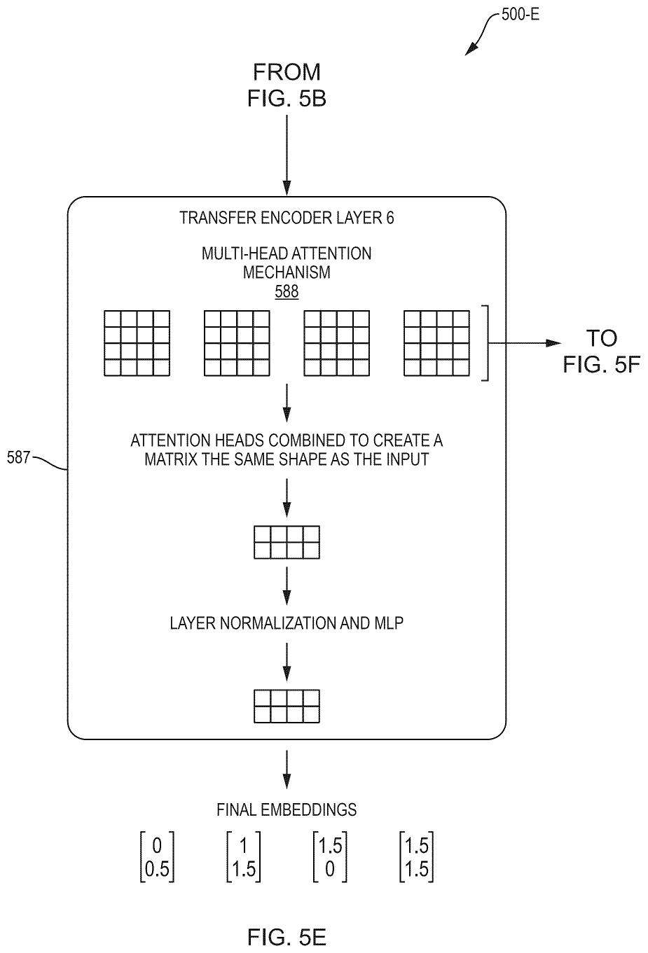

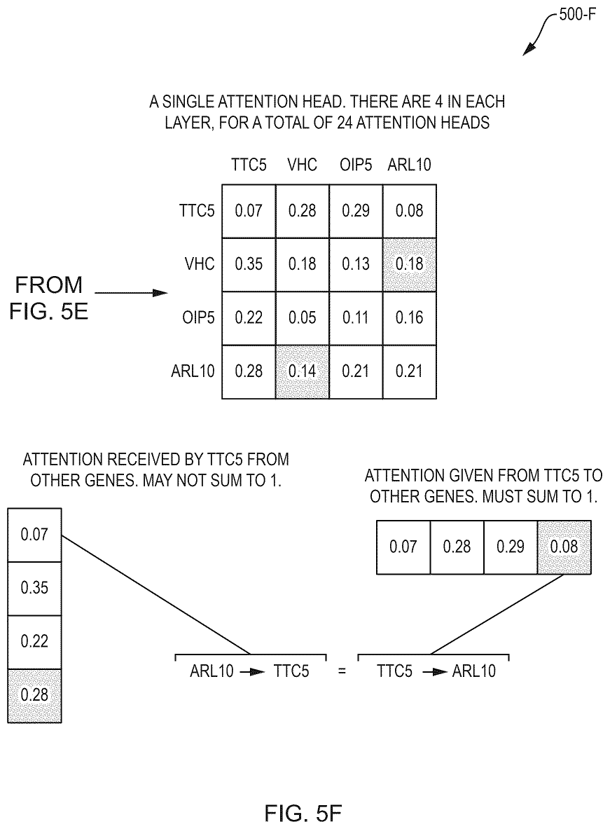

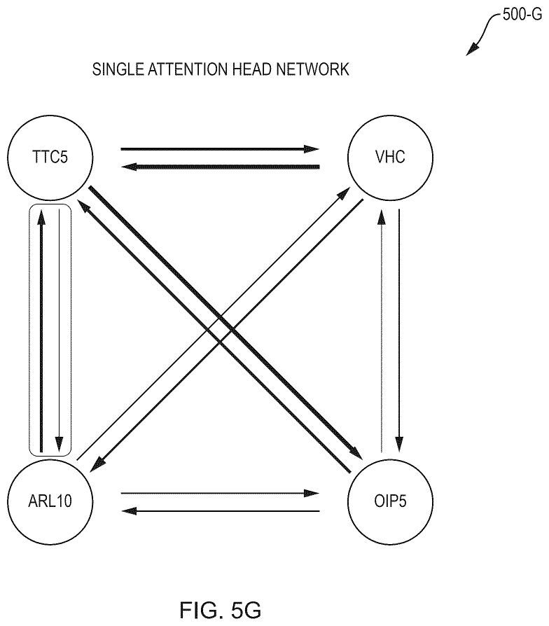

An example embodiment disclosed herein may be based on a hypothesis that GeneFormer's embeddings and attention weights will prioritize physical gene-gene interactions. Specifically, the maximum attention weight between genes i and j, max (Aᵢⱼ, Aⱼᵢ) over a large sample set should be larger if genes i and j are known to physically interact. The same should be true of the cosine similarity of the embeddings for genes i and j, they should be closer in embedding space if they are known to physically interact. To test this, the edges of a well validated Protein-Protein Interactome (PPI) were compared to all other possible interactions. In FIGS. 7A-C disclose that both attention weights and embeddings do show clear separation between PPI edges and other pairs of genes. As disclosed in FIGS. 7A-C, Geneformer prioritizes PPI edges.

FIG. 7A is a graph 700-A of an example embodiment of density 752-A vs. attention weight 754. Attention weights corresponding to PPI edges are higher than other attention weights.

FIG. 7B is a graph 700-B of an example embodiment of density 752-B versus cosine similarity 754. Cosine similarities between genes that have edges between them in the PPI are higher than cosine similarities between other pairs of genes. The PPI edge attentions and the background attentions are confirmed to come from different distributions using a KS-test. The PPI edge cosine similarities and the background cosine similarities pass the same test.

FIG. 7C is a graph 700-C of an example embodiment of a fraction of edges recovered 756 versus rank 758. Both ranked attention weights and ranked cosine similarities recover PPI edges faster than random expectation. Averaging an edge's ranking in the list of cosine similarities and the list of attention weights recovers edges at a similar rate as the ranked attentions alone.

According to FIGS. 7A-C, if one ranks the attention weights and cosine similarities, PPI edges may be recovered faster than would be expected from random selection. An average ranking was also tested, where edges are selected based on the average of their ranking in the cosine similarity list and the attention weight list. This yields a comparable recovery rate to attention weights alone.

### Fine-Tuning Geneformer Enhances Disease Modules

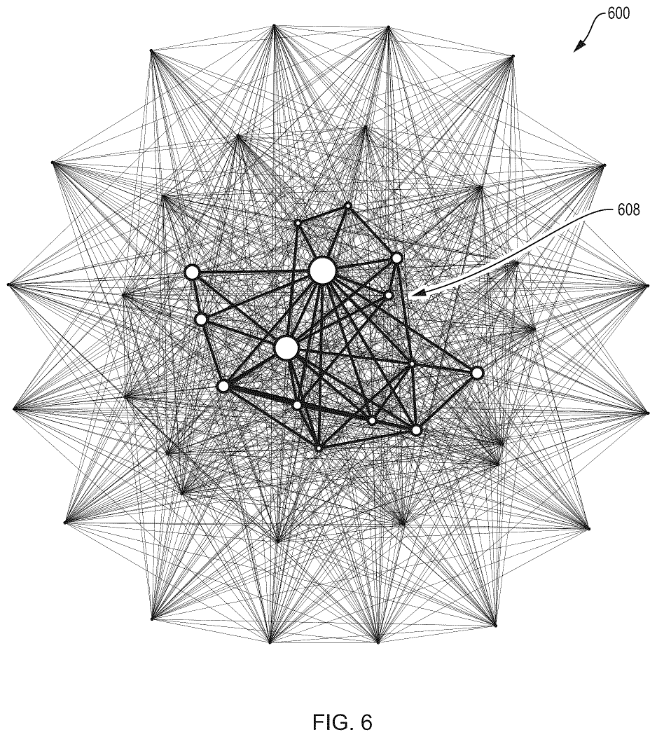

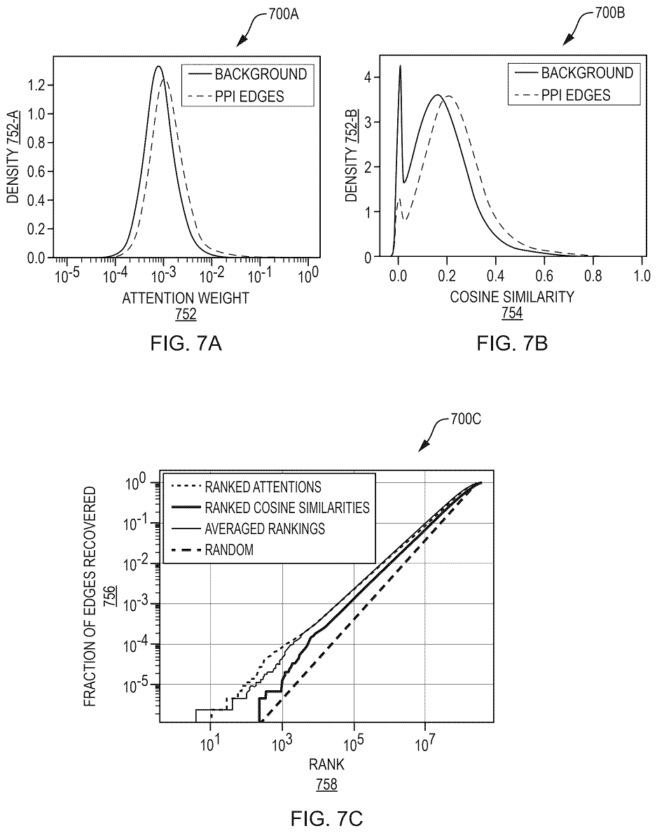

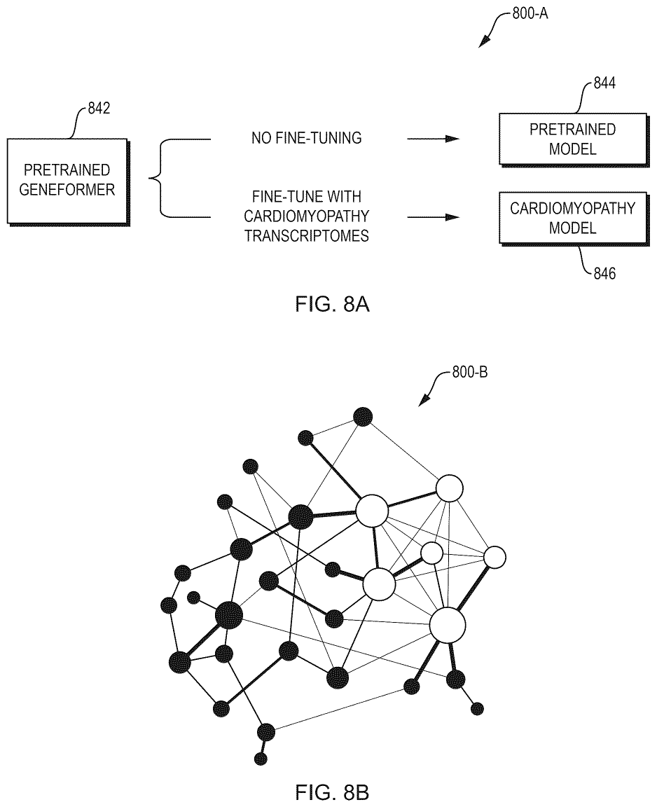

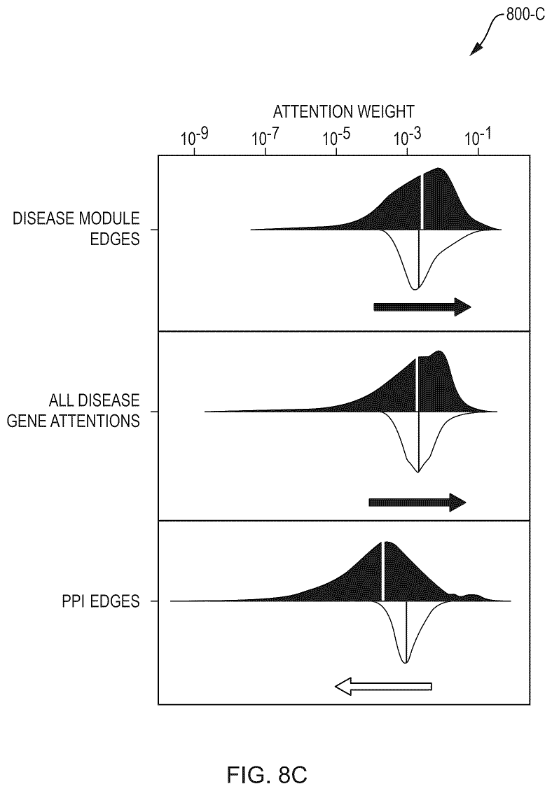

Given that the pretrained model clearly contains some information about the PPI, it was also hypothesized that fine-tuning the model should teach it something about disease modules. Disease modules are a network medicine tool used for drug re-purposing (Deisy Morselli Gysi, Ítalo do Valle, Marinka Zitnik, Asher Ameli, Xiao Gan, Onur Varol, Susan Dina Ghiassian, J. J. Patten, Robert A. Davey, Joseph Loscalzo, and Albert-László Barabási. Network medicine framework for identifying drug-repurposing opportunities for covid-19. Proceedings of the National Academy of Sciences, 118(19):e2025581118, 2021), defined as the largest connected component of known disease genes (genes that are known empirically to be related to a disease) in the PPI. If a Geneformer model has been fine-tuned on a specific disease, it was expected that the disease genes for that disease to be prioritized. It was further expected for those disease genes that are connected in the PPI to have even higher priority, because these are disease genes that interact in the context of a single cell. Such hypothesis was tested using an example embodiment of a fine-tuned cardiomyopathy model. Using 10,000 healthy heart cells (Nathan R. Tucker, Mark Chaffin, Stephen J. Fleming, Amelia W. Hall, Victoria A. Parsons, Kenneth C. Bedi, Amer-Denis Akkad, Caroline N. Herndon, Alessandro Arduini, Irinna Papangeli, Carolina Roselli, François Aguet, Seung Hoan Choi, Kristin G. Ardlie, Mehrtash Babadi, Kenneth B. Margulies, Christian M. Stegmann, and Patrick T. Ellinor. Transcriptional and cellular diversity of the human heart. Circulation, 142(5):466-482, 2020) the same attention weight aggregation was performed as performed to analyze PPI edges. Aggregate attention weights were aggregated from the pretrained model and the cardiomyopathy model, and the PPI edges were compared to edges in the disease module, and attention weights between disease genes that are not connected in the PPI. FIGS. 8A-D show that the fine-tuning process pushes attention weights between disease genes upwards and pushes disease module edges even further upwards. FIGS. 8A-D disclose disease modules in Geneformer.

FIG. 8A is a block diagram 800-A of an example embodiment of Geneformer 842 used both with additional fine-tuning (Cardiomyopathy model 844) and without (Pretrained model 846). The labels ‘pretrained’ and ‘cardiomyopathy’ refer to the model that was used to obtain the data.

FIG. 8B is an illustration 800-B of an example embodiment of a disease module in a network. Genes in light purple are disease genes, and edges in light purple are disease module edges. Light grey edges are interactions between disease genes that are not known to occur physically.

FIG. 8C is a table 800-C of an example embodiment of distributions of attention weights from the pretrained model and the cardiomyopathy model plotted for PPI edges, non-physical connections between disease genes, and disease module edges. PPI edges are shifted down during fine-tuning, while the other two sets are shifted up.

FIG. 8D is a table 800-D of an example embodiment of attention weights for both models that are plotted for several different disease modules. The top three diseases are heart diseases, and their disease module edges are pushed upwards by fine-tuning. The bottom three diseases are in other tissues, and their disease module edges are pushed down by fine-tuning.

An interrogation of how the fine-tuning process impacts other disease modules was performed. By comparing disease module edges in the pretrained model to the fine-tuned model, it was found that heart disease modules (cardiomyopathy dilated and cardiomyopathy hypertrophic, which were used to fine-tune the model, and heart failure, which was not) move up after fine-tuning. Non-heart diseases such as dementia, kidney failure, and rheumatoid arthritis move down after fine-tuning. It was concluded that fine-tuning the model on a disease in a specific tissue prioritizes disease modules from that tissue while deprioritizing disease modules from other tissues.

Finally, a test was performed on how quickly attention to disease genes drops off based on moving away from the disease module in the network. The network was separated into sets of genes that are 1, 2, 3, and 4 hops away from the disease module. It was found that 2 hops away from the disease module is indistinguishable from random in terms of the attention paid to disease module genes. Genes that are 3 and 4 hops away from the disease module have lower attention weights with the disease module than random genes. This aligns with network medicine understanding of disease modules, where genes 2 or more hops away are rarely considered for drug re-purposing because of the connectivity of the PPI. (Feixiong Cheng, Rishi J. Desai, Diane E. Handy, Ruisheng Wang, Sebastian Schneeweiss, Albert-László Barabási, and Joseph Loscalzo. Network-based approach to prediction and population-based validation of in silico drug repurposing. Nature Communications, 2018).

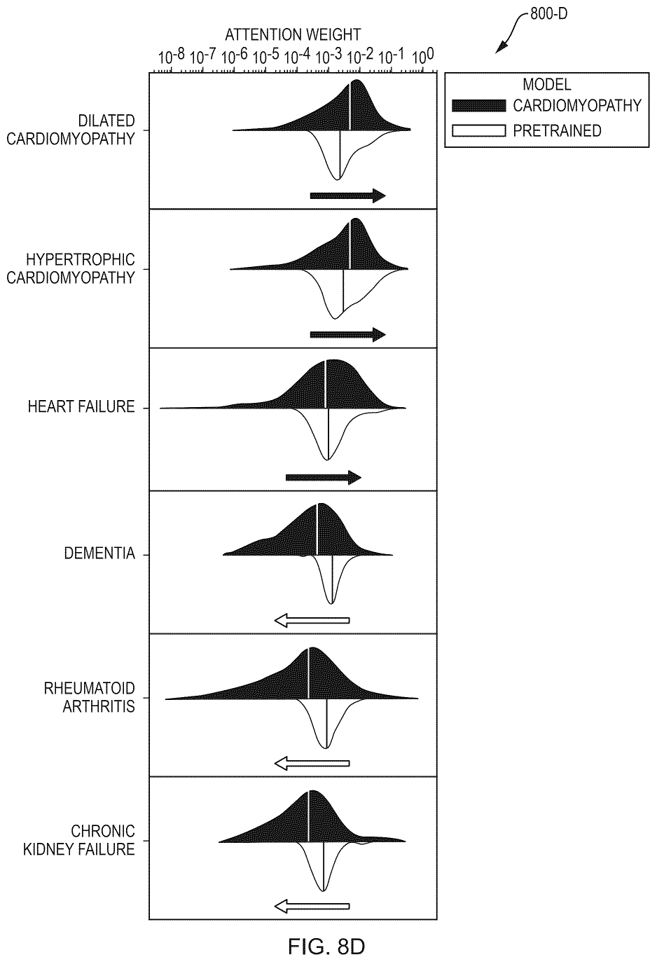

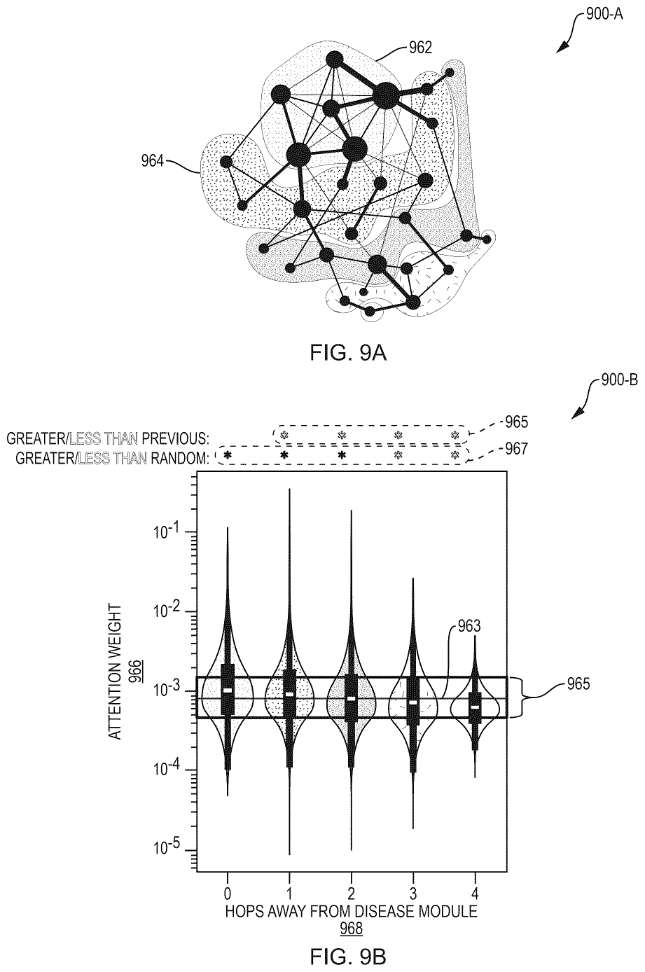

FIG. 9A is a diagram 900-A of an example embodiment of the disease module example from FIGS. 8A-D shaded to show different groups of nodes based on how many hops they are away from the disease module. The blue region 962 is the disease module, the orange region 964 is connected to the module by a single hop path, and so on.

FIG. 9B is a graph 900-B of an example embodiment of attention weight 966 versus hops away from the disease module 968. The distributions of attention weights between disease genes and genes that are n hops away from the disease module are plotted. The black line 963 and grey bar 965 are the mean and interquartile range for 100,000 randomly selected attention weights. The top row of stars 965 indicates that genes n+1 hops away have lower attention weights with the disease module than genes n hops away. The bottom row of stars 967 indicates that attention weights for genes that are 0, and 1 hops away from the disease module are greater than random, while 3 and 4 hops away are less than random. Attention weights for genes that are 2 hops away (which comprise a plurality of PPI genes) are indistinguishable from random.

### Geneformer Attention Weights Improve Disease Module Detection

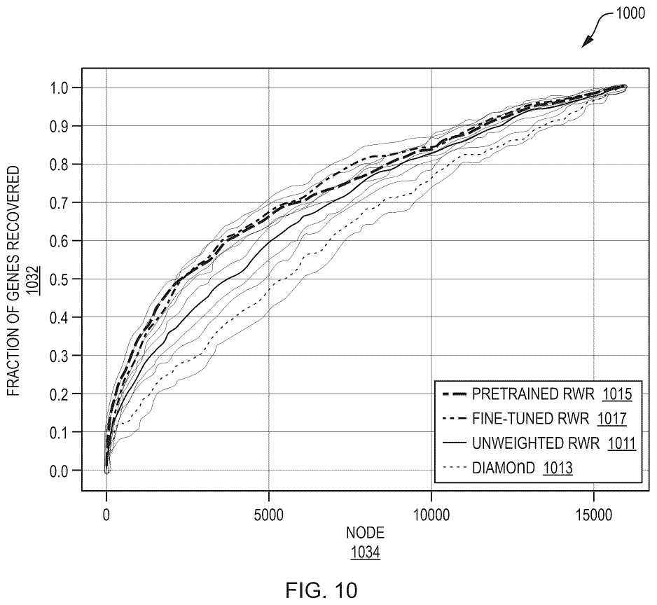

The capability of GeneFormer to improve upon current best practices for identifying candidate disease genes was tested. In order to identify new drug targets, it is often desirable to rank genes near the disease module in terms of their ‘closeness’ to the disease module. Because each gene in the PPI has on average NUMBER neighbors one cannot simply use shortest path lengths, there are too many candidate genes. Instead, an example embodiment used network methods such as random walk with restart (RWR) or DIAMOND (S. D. Ghiassian. A disease module detection (diamond) algorithm derived from a systematic analysis of connectivity patterns of disease proteins in the human interactome. PLOS Computational Biology, 11(4), April 2015). These methods rank genes according to the probability of visiting the gene (RWR), and the connectivity significance (DIAMOnD). While DIAMOND is performed on unweighted networks, RWR can be performed on weighted networks just as easily as unweighted. Therefore, each edge of the PPI was weighted with the maximum attention weight between each pair of genes in the 10,000 heart cell dataset (attention weights come from the pretrained model). FIG. 10 shows that this substantially boosts the predictive power of RWR, making the weighted RWR the best of the three methods for recovering disease genes. It is notable that the cardiomyopathy model is not necessary for this analysis. While the cardiomyopathy attention weights boost performance in the later portion of the analysis, the first several thousand genes (which would contain all candidate disease genes) remain unchanged. This means that rather than requiring 100,000 cells to fine-tune a model on a specific disease, this analysis only requires 10,000 healthy cells from the relevant tissue.

FIG. 10 is a graph 1000 of an example embodiment of a fraction of genes recovered 1032 per node 1034. The graph 1000 discloses that Geneformer attention weights improve capture-recapture analysis for disease module discovery. Capture-recapture analysis is performed on the unweighted PPI (unweighted RWR 1011, DIAMOND 1013) as well as a PPI weighted with attention weights from the pretrained model (pretrained RWR 1015) and the cardiomyopathy model (fine-tuned RWR 1017). Both weighted networks outperform the unweighted, but the pretrained and fine-tuned model perform similarly.

### Geneformer Attention Weights Improve Drug Repurposing

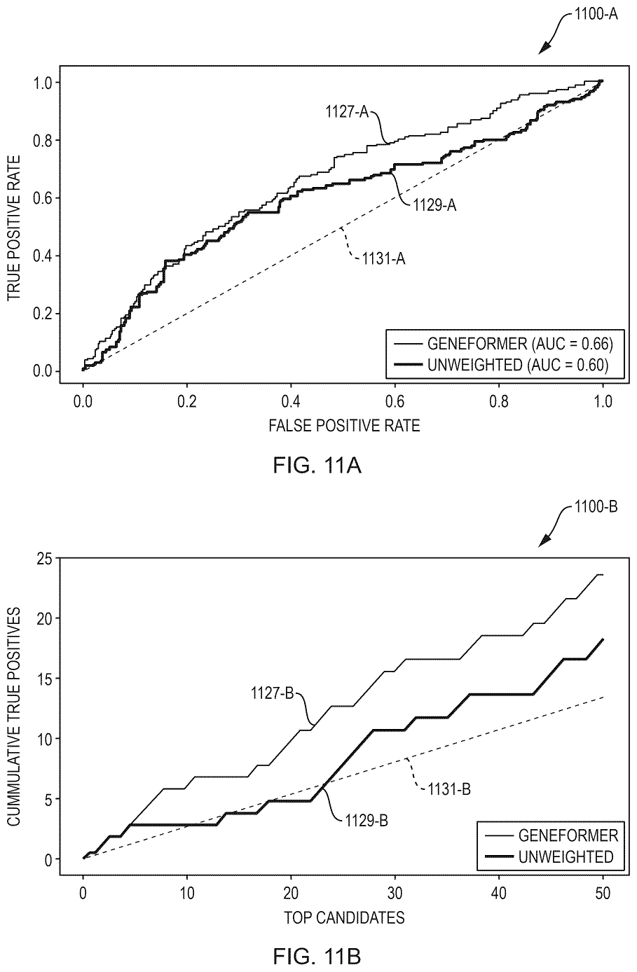

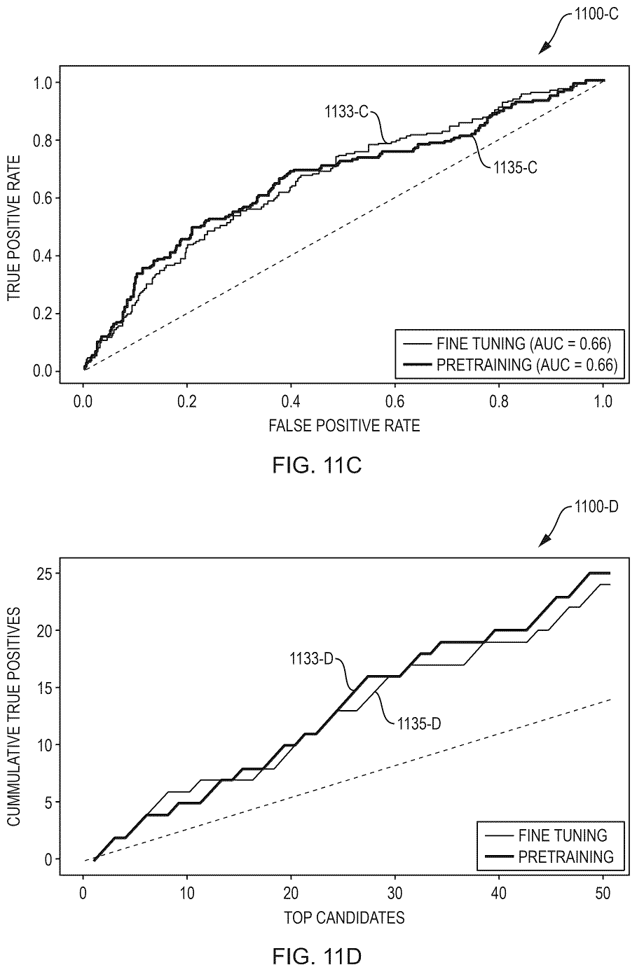

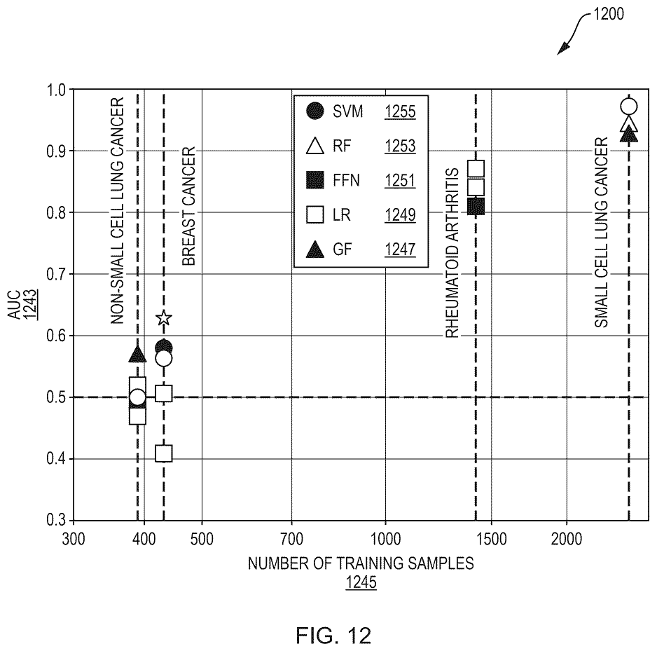

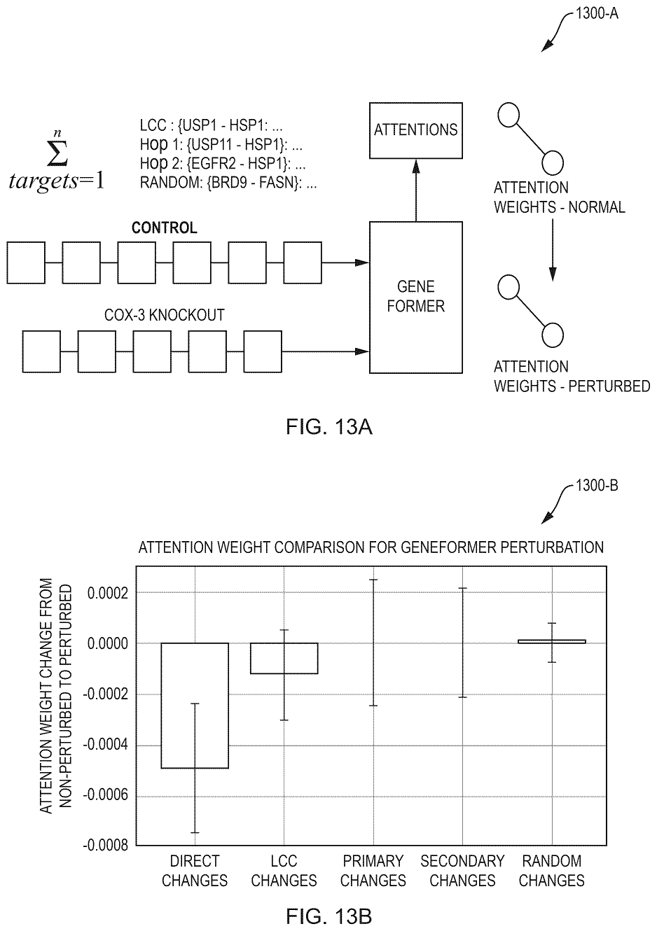

Next, an evaluation was performed for whether the attention weights of Geneformer could assist in drug repurposing studies to predict potential treatments for a specific disease. First, Geneformer was fine-tuned to distinguish between cardiomyocytes from healthy hearts and those affected by dilated cardiomyopathy (DCM), achieving an overall accuracy of (NUMBER). The attention weights from the Geneformer DCM model were then extracted and used to assign weights to the protein-protein interaction (PPI) network. Subsequently, a drug repurposing study was performed by calculating network-based proximity scores between the DCM disease genes and the targets of 618 drugs, including 171 DCM-related (Positive) and 447 unrelated (Negative) drugs extracted from DRUGBANK (see Methods). Lower proximity scores indicate shorter distances between the drug targets and the DCM genes. The drug list as ordered based on their corresponding scores and the number of positive and negative drugs predicted by the DCM-Geneformer weighted network was tracked. The results showed that the Geneformer weighted PPI network improved the overall prediction capabilities for identifying DCM-related prescribed drugs, achieving an AUC of 0.66 compared to an AUC of 0.60 with the unweighted PPI, as disclosed in FIG. 11A. Additionally, the number of Positive and Negative drugs among the top 50 predicted candidates was assess. It was found that the Geneformer weighted PPI model consistently outperformed the unweighted PPI model, identifying a larger set of prescribed drugs for DCM, as disclosed in FIG. 11B). Finally, an investigation was performed into whether the DCM fine-tuning contributed to reinforcing the PPI weights compared to a PPI network weighted with pre-trained Geneformer attention weights for the drug repurposing study. Little difference was observed between the PPI networks weighted with the fine-tuned and pre-trained models, as disclosed in FIGS. 11C and 11D, below. FIGS. 11A-D shown that Geneformer attention weights enhance drug repurposing for dilated cardiomyopathy (DCM).

FIG. 11A is a graph 1100-A of an example embodiment of receiver operating characteristic (ROC) curves. In the graph 1100-A, ROC curves compare the Geneformer model's weighted PPI network (red) 1127-A with the unweighted PPI network (black) 1129-B in identifying DCM-related drugs. The Area Under the Curve (AUC) values are 0.66 and 0.60 for the Geneformer and unweighted models, respectively. The dashed black line 1131-A represents a random classifier.

FIG. 11B is a graph 1100-B of an example embodiment of cumulative true positives among the top 50 drug candidates predicted by proximity scores for the Geneformer weighted PPI network (red) 1127-B compared to the unweighted PPI network (black) 1129-B. The dashed line 1131-B indicates random selection.

FIG. 11C is a graph 1100-C of an example embodiment of ROC curves for the Geneformer weighted PPI network (orange) 1135-C and a PPI network weighted using a pre-trained Geneformer model (blue) 1133-C. Both models achieve an AUC of 0.66.

FIG. 11D is a graph 1100-D of an example embodiment of cumulative true positives among the top 50 drug candidates for the Geneformer weighted PPI network (orange) 1135-D and the pre-trained Geneformer model weighted PPI network (blue) 1133-D. The dashed line represents a random selection.

### GeneFormer can be Trained on Bulk RNA

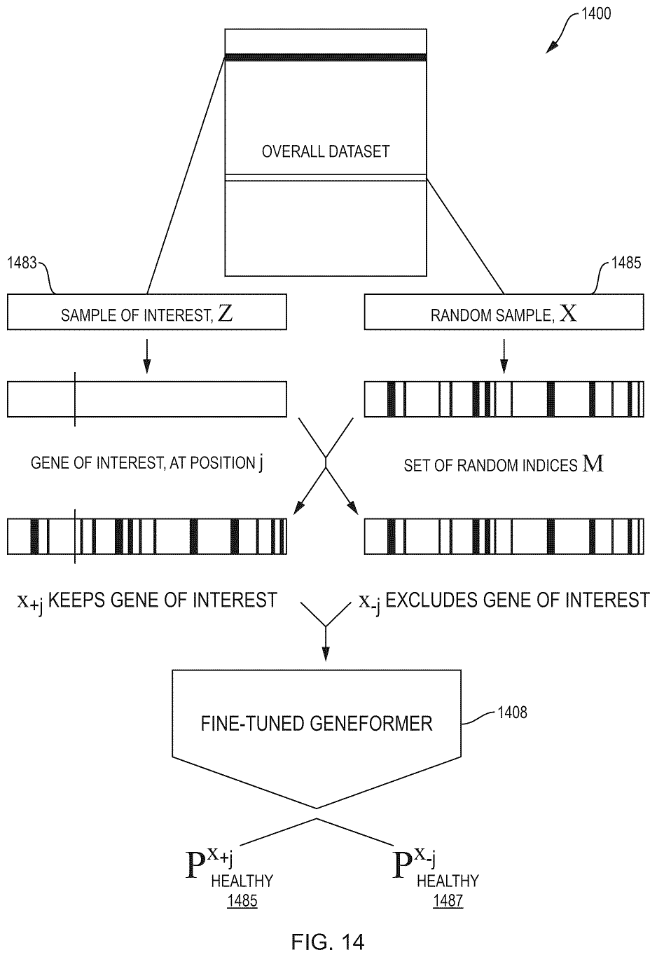

A test was performed for whether Geneformer, which has been pretrained on single cell data, can be fine-tuned on bulk RNA data to predict disease states. Using 4 datasets from GEO, which contain disease samples and healthy controls for rheumatoid arthritis (RA-MAP Consortium. Ra-map, molecular immunological landscapes in early rheumatoid arthritis and healthy vaccine recipients. Sci Data, 9(1):196, May 2022. PMID: 35534493), breast cancer (Su Bin Lim. A microarray meta-dataset of breast cancer. BioStudies, E-MTAB-6703, 2019. Retrieved from www.ebi.ac.uk/biostudies/arrayexpress/studies/E-MTAB-6703), small cell lung cancer (Su Bin Lim. A microarray meta-dataset of small cell lung cancer. BioStudies, E-MTAB-6699, 2019. Retrieved from www.ebi.ac.uk/biostudies/arrayexpress/studies/E-MTAB-6699), and non small cell lung cancer (Su Bin Lim. A microarray meta-dataset of non-small cell lung cancer. BioStudies, E-MTAB-6043, 2018. Retrieved from www.ebi.ac.uk/biostudies/arrayexpress/studies/E-MTAB-6043), 4 versions of Geneformer were fine-tuned, namely, a basic random forest, support vector machine, feed forward network, and logistic regression on the same data. It was found that Geneformer, while competitive with the other models, does not outperform the more basic machine learning methods on bulk RNA data. The poor predictive power in the breast cancer and non-small cell lung cancer samples may be attributed to the small sample size of the data. As shown in FIG. 12, disclosed below, the predictive power of all the models increases with increasing training data, and those two datasets have very limited control sample sets.

FIG. 12 is a graph 1200 of an example embodiment of area under curve (AUC) values for benchmark numbers of training samples 1245 for different models. The graph 1200 shows results for training Geneformer (GF) 1247 on bulk RNA. Geneformer is trained on bulk data to diagnose four diseases, and compared to other basic machine learning models, random forest (RF) 1253, support vector machine (SVM) 1255, feed forward network (FFN) 1257 and logistic regression (LR) 1249. Geneformer (GF) 1247 is competitive with other methods but does not outperform them on bulk data as it does on single cell data. A clear sample size effect is observed on all models. As the size of the dataset (listed next to the disease on the x axis) increases, so does the AUC 1243 of all five models. The x-axis is log-scaled, with ticks to point out benchmark sample sizes.

### Insights

Transformer models, originally showing state-of-the-art performance in NLP tasks, have been successfully applied to a variety of domains, including cell-level and gene-level classification. Internal embedded model states of traditional fully-connected networks have been used for biological interpretability in many contexts. An example embodiment disclosed herein is based on a theory that the attention mechanisms of transformers can likewise be extrapolated for interpretability in a biological network-based context.

It has been demonstrated that the pretrained Geneformer prioritizes known physical gene-gene interactions, and fine-tuning the model on a disease further emphasizes disease gene relationships. As disclosed herein, by using only the pretrained model and a dataset of healthy single cells from a tissue of interest, disease module detection can be improved over existing network medicine methods. Further, it is disclosed herein that GeneFormer can also be trained on bulk RNA data, reinforcing the idea that it has learned relationships that transcend the single cell level.

### Methods

### Classifying Genes and Interactions

For attention weight analyses, genes were divided into four classes. PPI genes are genes that appear in our protein-protein interactome, which has 17,530 genes and 503,177 edges (A.-L. Barabási, N. Gulbahce, and J. Loscalzo. Network medicine: a network-based approach to human disease. Nat Rev Genet, 12(1):56-68, 2011). Background genes are genes that do not appear in the PPI, which make up 7,894 of GeneFormer's 25,424 gene vocabulary. Disease genes are genes that appear in the PPI and are also experimentally shown to be associated with a disease of interest (Deisy Morselli Gysi, Ítalo do Valle, Marinka Zitnik, Asher Ameli, Xiao Gan, Onur Varol, Susan Dina Ghiassian, J. J. Patten, Robert A. Davey, Joseph Loscalzo, and Albert-László Barabási. Network medicine framework for identifying drug-repurposing opportunities for covid-19. Proceedings of the National Academy of Sciences, 118(19):c2025581118, 2021). Disease module genes are the largest connected component of disease genes in the PPI. If a disease has 150 genes associated with it, but only 148 of them appear in the PPI, and only 120 of those are connected in the PPI, resulting in a group of 148 disease genes and 120 disease module genes.

Because attention weights correspond to edges, not nodes, the edges are also divided into four classes. The first is PPI edges. The second is background edges, which are any edges that do not occur in the PPI. The genes on either end may both be PPI genes, but if the edge does not exist in the PPI it is a background edge. Disease module edges are PPI edges that connect two disease module genes. The ‘fully-connected disease module’ refers to all other possible edges between disease genes. These edges are a subset of the background.

Aggregating Weights from Geneformer

To compare attention weights between different sets of genes, we begin by selecting a set of 10,000 samples. For FIGS. 7A-C, which tests the PPI edges against the background of all other possible interactions, we use 10,000 random samples from the Genecorpus pretraining dataset. For FIGS. 8A-D and 9A-B, which compare heart disease genes to other sets of genes, 10,000 healthy heart cells were used from the training data for the Geneformer author's cardiomyopathy training data (Nathan R. Tucker, Mark Chaffin, Stephen J. Fleming, Amelia W. Hall, Victoria A. Parsons, Kenneth C. Bedi, Amer-Denis Akkad, Caroline N. Herndon, Alessandro Arduini, Irinna Papangeli, Carolina Roselli, François Aguet, Seung Hoan Choi, Kristin G. Ardlie, Mehrtash Babadi, Kenneth B. Margulies, Christian M. Stegmann, and Patrick T. Ellinor. Transcriptional and cellular diversity of the human heart. Circulation, 142(5):466-482, 2020). These samples were then passed through a Geneformer model, either the pretrained model with no fine-tuning, or the cardiomyopathy model provided by the Geneformer authors. For each sample, the four attention heads were extracted from the second to last layer of the model. A single layer was selected because the overall distribution of attention weights shifts considerably from layer to layer. The second to last layer was selected on the recommendation of the Geneformer authors, who observe that the attention weights from the final layer are task specific.

After extracting the attention heads from the second to last layer, the max (Aᵢⱼ, Aⱼᵢ) across all four heads for each pair of genes is taken, giving a single attention weight for each pair of genes in the sample. This maximum aggregation was performed across each sample in the data, resulting in a large matrix of the maximum attention weight between every pair of genes that occur in the data. This matrix can then be filtered for certain subsets of gene-gene interactions, such as PPI edges.

### Capture-Recapture Analysis of Disease Modules

The aggregated attention weights from the healthy heart cells are used to create a weighted PPI. To do this, some edges are removed from the PPI, as some pairs of genes never occur together in the 10,000 cell dataset. The resulting PPI has NUMBER nodes and NUMBER edges, approximately 85% of the original PPI in both cases. The cardiomyopathy disease module in this network has NUMBER nodes and NUMBER edges, which is approximately 95% of the original disease module. The edges of this disease module are then weighted using the aggregated attention weights.

Starting with the cardiomyopathy disease module, half of the disease genes to keep are selected randomly. These genes are used as seed genes for network's pagerank algorithm. A vector of probabilities is created so that the probability of restarting from a seed gene is n−1 and the probability of restarting from any other node is 0.

This modified pagerank was run with a restart probability of 0.4, and this process was repeated 10 times with different randomly selected seeds. In FIG. 10, the resulting list of genes is plotted, ranked by probability of visitation. The ribbon is the standard deviation across the 10 runs. This process was repeated with the unweighted network. DIAMOND 1013, which is only run on the unweighted network.

### Drug Repurposing Studies

Geneformer was fine-tuned to distinguish between cardiomyocytes from healthy hearts and those affected by dilated cardiomyopathy (DCM) using the original Geneformer Cardiomyopathy dataset (C. V. Theodoris et al. Transfer learning enables predictions in network biology. Nature, 618(7965):616-624, June 2023).

After fine-tuning, the attention weights from the final layer of the Geneformer DCM model were extracted and aggregated according to the procedure disclosed above with reference to aggregating weights from Geneformer. These aggregated weights were assigned to the links in the protein-protein interaction (PPI) network. For the pre-trained weighted PPI, a similar procedure was followed, extracting the attention weights from the final layer of the pre-trained Geneformer model.

The dilated cardiomyopathy (DCM) disease genes were identified and then drug targets were extracted from DRUGBANK (Craig Knox, Michael Wilson, Christopher M Klinger, et al. Drugbank 6.0: the drugbank knowledgebase for 2024. Nucleic Acids Research, 52(D1):D1265-D1275, 2024) for 618 drugs, comprising 171 drugs currently approved for treating DCM-related conditions (Positives) and 447 drugs not used for DCM (Negatives). The Positive drugs included 7 for dilated cardiomyopathy, 1 for ischemic heart disease, 121 for hypertension, 66 for heart failure, and 20 for arrhythmia. The negative drugs were selected as those that were neither approved nor under investigation for DCM treatment and whose targets were not found among the DCM disease genes.

A proximity score (Emre Guney, Jörg Menche, Marc Vidal, et al. Network-based in silico drug efficacy screening. Nat Com-mun., 7:10331, 2016) was calculated for each of the 618 drugs, measuring the network distance between the DCM disease genes and the drug targets. Lower proximity scores indicate closer proximity between drug targets and DCM genes. The drugs were then ranked based on their proximity scores. The predictive performance of the Geneformer weighted PPI network was evaluated using these ranked proximity scores to generate receiver operating characteristic (ROC) curves and calculate the area under the curve (AUC). Additionally, the number of prescribed (Positive) drugs identified among the top N candidates was assessed.

### Training on Bulk RNA

To train Geneformer on bulk RNA data, 4 datasets were downloaded from GEO which contain both disease and control samples for four diseases. The expression data is tokenized, and a subset used to create balanced classes for training. Then 80/20 test splits were created and Geneformer was trained on the bulk data. This process was repeated 3 times because the small number of samples creates variability in the resulting AUC. Basic ML models were also trained on the raw counts. For all models besides Geneformer, sklearn was used for default training parameters (except for random forest, where the max depth is set to 2). For Geneformer, hyperparameters from the fine-tuning examples on the Geneformer huggingface repo were used. FIG. 12 shows the mean AUC of each model on the test sets. Table 1, disclosed below, lists data volume for five different datasets containing transcriptomes of disease patients (Case) and healthy controls (Control). Geneformer was fine-tuned on each dataset to identify transcriptomes of disease patients.

TABLE 1

| | | Non-Small | Small | |
|---|---|---|---|---|
| | | Cell | Cell | |
| | Breast | Lung | Lung | Rheumatoid |
| Dataset | Cancer | Cancer | Cancer | Arthritis |
| Total data | 2,302 | 1,118 | 2,739 | 1,366 |
| Case/Control | 2,088/214 | 925/193 | 1,474/1,265 | 1961/707 |
| Balanced Sample | 214-214 | 193-193 | 1,265-1,265 | 707-707 |
| Final data size | 428 | 386 | 2,530 | 1414 |
| Tissue | mammary | lung | lung | whole-blood |

### Supplementary Method and Results

### Gene Perturbation Impact on Attention Weights

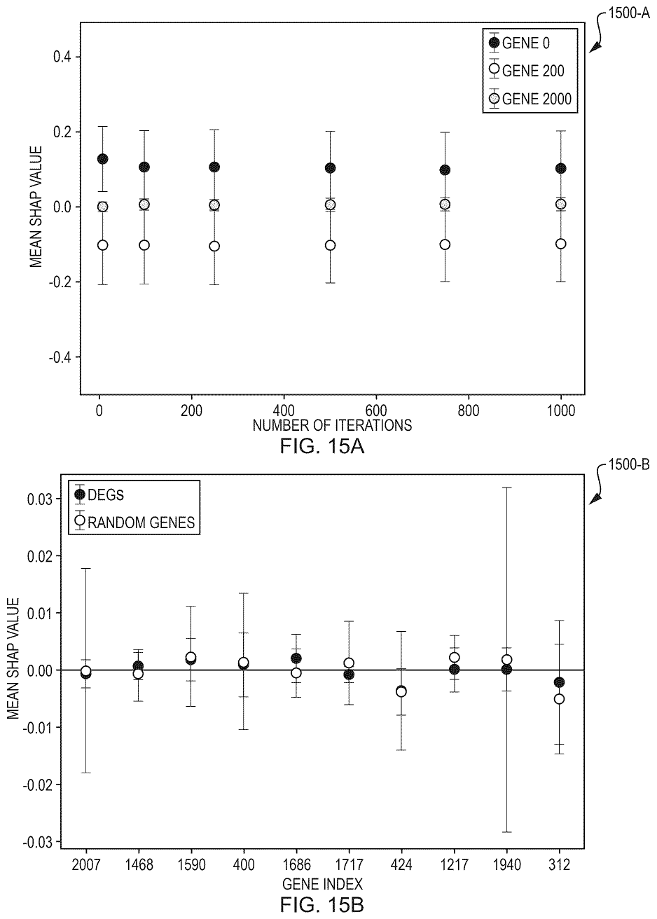

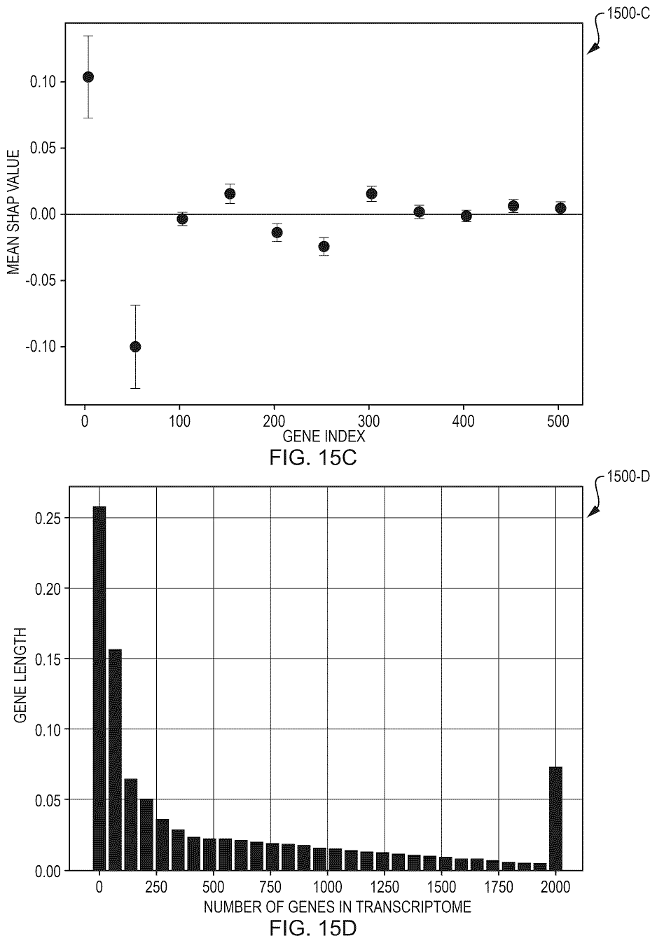

Gene perturbation was evaluated as another potential mechanism for interpretation of Geneformer attention weights. The largest connected component of the cardiomyopathy disease module was removed from the training transcriptomes, and the attention weights of an aggregated dataset containing 1,000 samples with the node included as a tokenized gene was compared to the perturbed dataset as disclosed in FIGS. 13A and 13B below. The largest change in the attention weights consisted of edges directly connected to the LCC, with a significant difference for edges within the LCC. Certain genes one or two hops away from the LCC displayed significant differences compared to the aggregate change of the edges outside of the LCC.

FIG. 13A is a block diagram 1300-A of an example embodiment of an attention weight comparison method.

FIG. 13B is a graph 1300-B of an example embodiment of comparison weights for direct edges from perturbed genes, edges in the disease LCC, edges 1 and 2 hops from the LCC, and background edges. FIGS. 13A and 13B show change between perturbed and control embeddings for network derivatives.

### Shapley Approximation

To analyze feature importance in Geneformer, a Monte-Carlo approximation of Shapley values was implemented. Shapley values are a well-known game theory technique for assigning the contribution of each ‘player’ in a ‘coalition’ to the total output of the coalition. In this case the coalition is a sample, and the players are genes. Shapley values take O(2^{N}) to compute exactly but can be approximated using the following method.

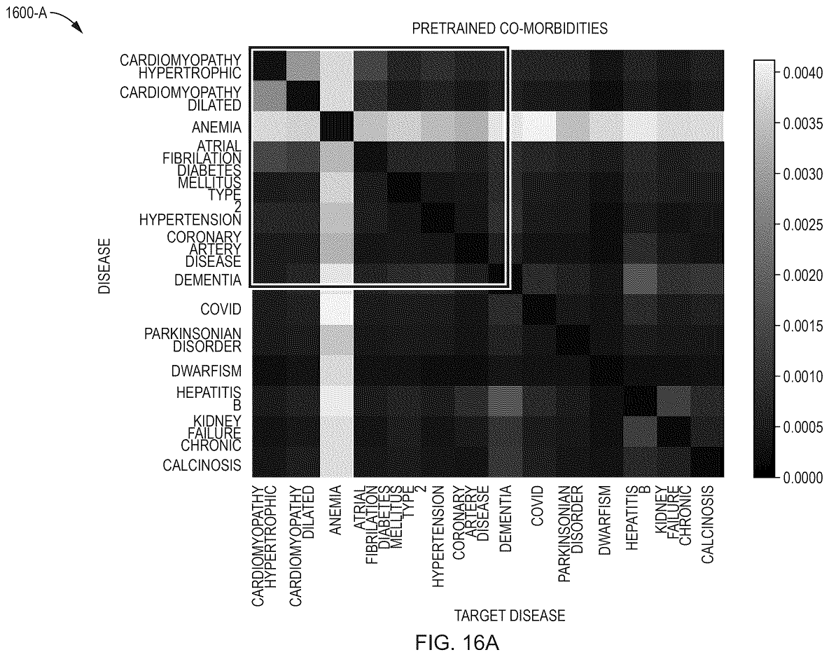

Given a dataset such as RA-map, first pick a sample of interest, which is called Z. Then pick a gene to explain, which has index j in sample Z. Select another random sample, X, and pull a random set of indices from that sample (between 20% and 80% of the sample). Such indices are called indices M, such that the genes in sample X corresponding to indices M are given by X_{M}. Then two synthetic samples called x+j and x−j are created, and x−j is created by replacing Z_{M} with X_{M}, x+j is created by replacing Z_{M} with X_{M} and replacing Zⱼ with Xⱼ. This creates two unique, synthetic samples, that are identical except at position j, where only one of the samples has the gene of interest. This process is illustrated in FIG. 14.

FIG. 14 is a flow diagram 1400 of an example embodiment of a single iteration of a Shapley approximation. Starting from a sample of interest 1483 and a random sample 1485, two fake samples are created. These samples are identical except that one contains the gene of interest. These samples are then run through the model 1408, which returns the probability that each sample is healthy. The difference between these probabilities (1485, 1487) is the marginal contribution of a single iteration, and the Shapley value is approximated as the average of many marginal contributions.

With the synthetic samples made, both samples were run through the relevant Geneformer model (in this example where the dataset is RA-map, the model would be the fine-tuned model used to classify bulk RNA samples for RA). This model is a binary classifier, so the difference in the predicted probability of the sample being a healthy sample between the two synthetic samples is measured. This gives a single measurement of the probability added by the gene of interest. This process is repeated 500 times, and the average value is taken. While these 500 iterations are far less than the 22048 iterations used to compute the exact Shapley value, it was found that the results of the approximation converge after 500 iterations. Using this process the Shapley values for genes at different locations in the rank value encoding is plotted, as disclosed below, finding that the influence on the outcome drops very quickly. By the time the 500th gene in the transcriptome is reached, the influence on the outcome is indistinguishable from 0.

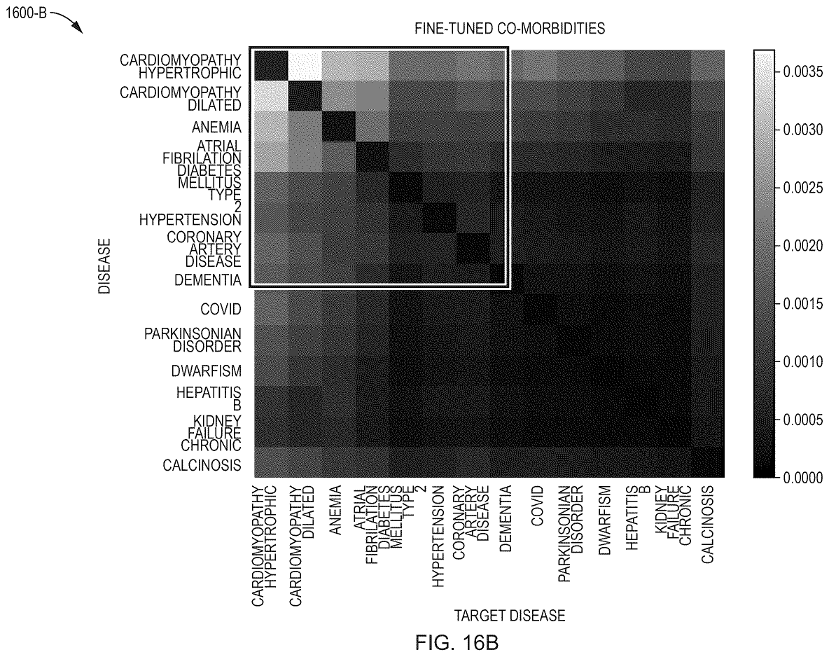

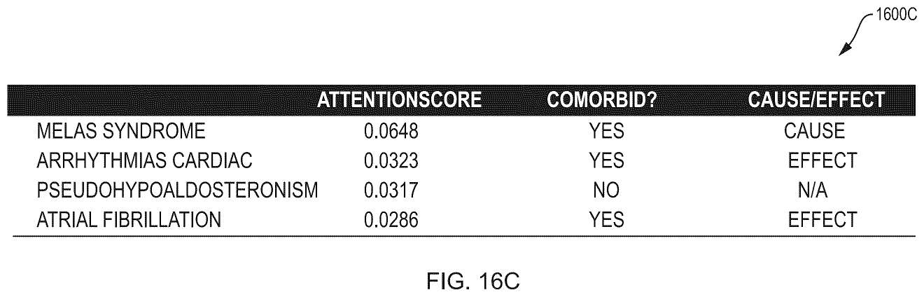

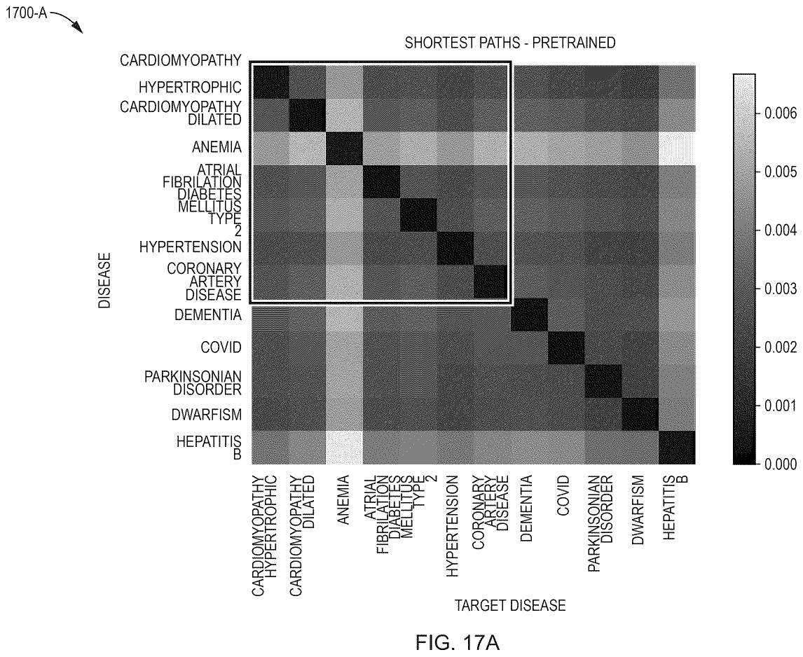

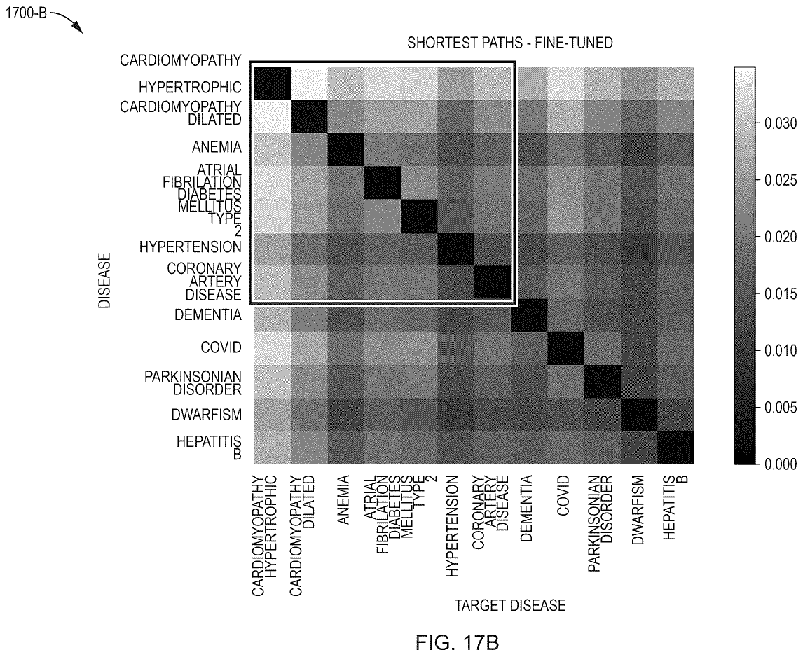

FIG. 15A is a plot 1500-A of an example embodiment of Shapley values per number of iterations for different genes, namely gene 0, gene 200, and gene 2000. It was found that as the number of iterations is increased past 500, the average of the marginal contributions remains stable. For the rest of this analysis, 500 iterations are used to approximate the Shapley value.

FIG. 15B is a plot 1500-B of an example embodiment of the Shapley value of genes at different positions in a rank value encoding. It is found that the contribution of each gene to the model's decision drops off very quickly, nearing 0 by the time position 500/2048 is reached.

FIG. 15C is a plot 1500-C of an example embodiment of the Shapley value for 10 genes that are over expressed in disease samples, and 10 genes that occur at the same position as the differentially expressed genes (DEGs), but in random samples.

FIG. 15D is a plot 1500-D of an example distribution of transcriptome lengths in Geneformer's pretraining corpus.

With reference to FIGS. 15A-D, a disease sample was picked and the Shapley values plotted for 10 genes that are over-expressed in disease samples. For comparison, another random disease sample was selected, and the Shapley values plotted for the same positions in the transcriptome (ensuring that none of those genes are differentially expressed). In this way a test is performed for whether the influence on the outcome is due to the fact that the gene is differentially expressed or the fact that it occurs at a given location in the transcriptome. It was found that the randomly selected genes track very closely with the differentially expressed genes at the same location, suggesting that the identity of the gene has much less impact on the result than the expression of that gene (and therefore its location in the rank-value encoding).

It is notable that the Shapley values reach 0 after a few hundred genes, because that is the length of over 60% of the samples in Geneformer's pretraining corpus. It is possible that Geneformer has been trained to assign more importance to the first few hundred genes because most samples it sees during pretraining do contain all relevant information in these indices. This implies that bulk samples, which include thousands more genes than single cell samples, may be introducing too much noise to be effectively leveraged by Geneformer.

### Comorbidities

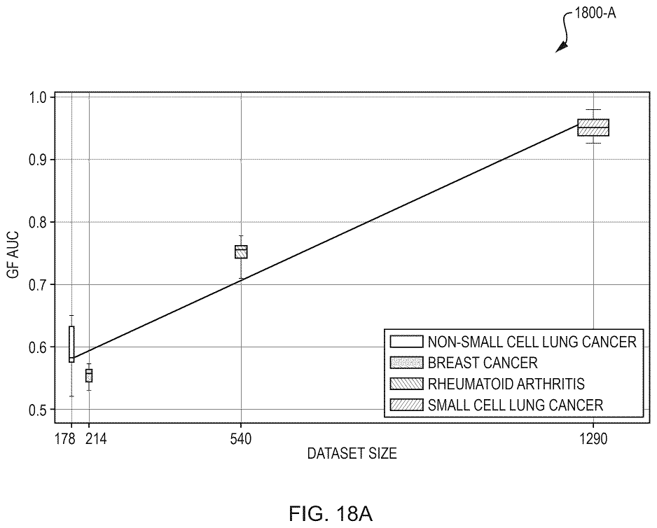

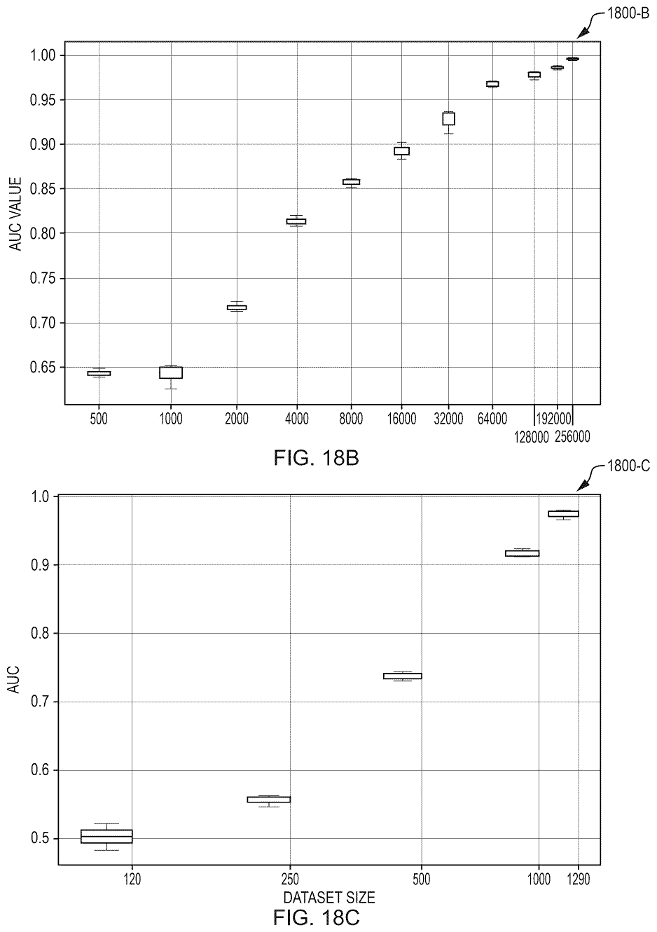

Geneformer-based attention weights were applied to properly identify disease comorbidities. The maximum attention weights from direct connections between every source gene in a given disease LCC to every target gene in a target disease LCC were aggregated together to create a direct correlation score between the source and target disease module. Cardiomyopathy dilated, anemia, atrial fibrillation, diabetes mellitus type 2, hypertension, and coronary artery disease were selected as co-morbid diseases (E. M. S. et al., Clin Res Cardiol 112, 123 (2023), and dementia, COVID, hepatitis B, Parkinsons, calcinosis, dwarfism, and kidney failure were selected as non-comorbid diseases. The fine-tuned cardiomyopathy model was used as a substrate for analysis compared to the pretrained model, with heat maps of the scores between a variety of comorbidities and unrelated diseases tested. Heat maps were then plotted of the scores between each of the disease modules to every other disease module, as disclosed in FIGS. 16A and 16B.

FIGS. 16A and 16B are example embodiments of heat maps 1600-A and 1600B, respectively, for an attention-based comorbidity analysis for cardiomyopathy. FIG. 16A shows comorbidity analysis using direct comparison of gene-gene pretrained Geneformer weights. FIG. 16B shown comorbidity analysis using direct comparison of gene-gene fine-tuned Geneformer weights.

FIG. 16C is a table 1600-C of name and comorbidity status of top 4 disease-disease interactions by ranked direct attention weight (Y.-H. R. Hsu, H. Yogasundaram, N. Parajuli, L. Valtuille, C. Sergi, and G. Y. Oudit, Heart Failure Reviews 21, 103 (2016), ISSN 1573-7322, URL https://doi.org/10.1007/s10741-015-9524-5, (J. R. Buckley, S. L. Harrison, D. Gupta, E. Fazio-Eynullayeva, P. Underhill, and G. Y. H. Lip, JAHA 10, c021970 (2021) for cardiomyopathy hypertrophic.

With reference to FIGS. 16A and 16B, the pretrained model heat map shows little to no correlation between cardiomyopathy and its comorbidities, as well as any of the comorbidities to one another. Once fine-tuned, the connections between cardiomyopathy and it's comorbidities increases significantly, with lesser increases seen between the comorbidities and other comorbidities. Only the scores indicating the relationships between diseases that were not comorbid decreased or remained relatively unchanged. The direct scores between the cardiomyopathy hypertrophic LCC and LCCs from over 300 other GDA modules were sorted by score. Of the top four ranked disease modules, cardiac arrhythmia and atrial fibrillation are directly co-morbid with cardiomyopathy, while research has shown in recent years that Melas syndrome (mitochondrial dysfunction) has been correlated with cardiomyopathy and heart failure (Y.-H. R. Hsu, H. Yogasundaram, N. Parajuli, L. Valtuille, C. Sergi, and G. Y. Oudit, Heart Failure Reviews 21, 103 (2016), ISSN 1573-7322, //doi.org/10.1007/s10741-015-9524-5), illustrating the potential use of attention-based scoring mechanisms for discovery and/or validate of disease comorbidity predictions.

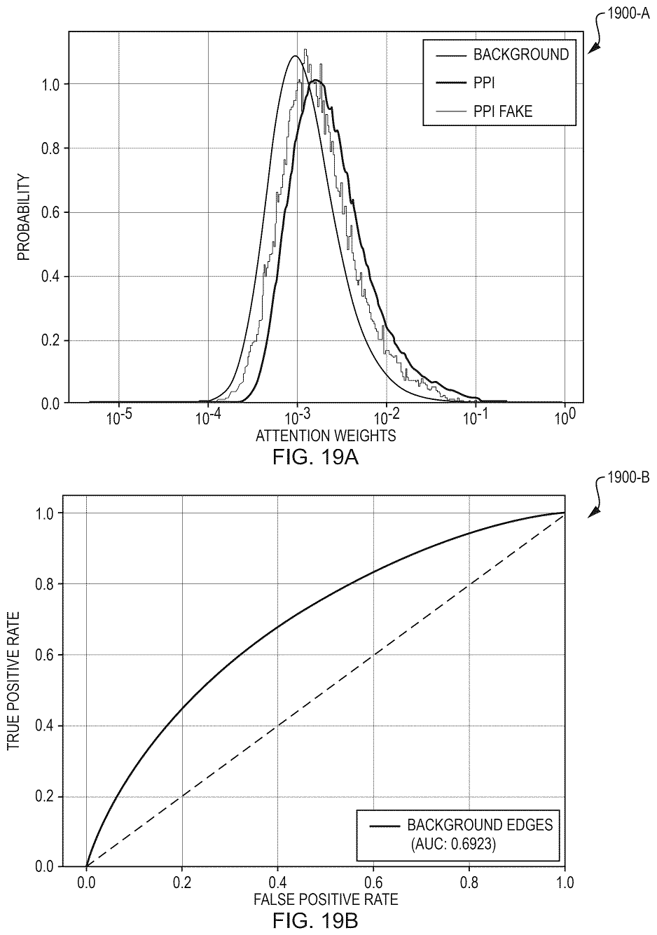

A secondary method was developed to assess disease comorbidities utilizing attention weights. This method involved finding the shortest path from every gene in one disease LCC to the comparison disease LCC on an interactome weighted with aggregate attention weights. The average weight of all the edges in the shortest path was aggregated for each node, and then the average of that value was taken as the score for the interaction between that disease module and the target disease module. The results disclosed in FIGS. 17A and 17B indicate a clear increase in signal for the comorbidities for cardiomyopathy dilated and hypertrophic compared to the background when using the methodology, but the correlation and net change was less significant than utilizing the direct method, where the direct weights from each disease gene to each other disease gene were used as a metric.

FIGS. 17A and 17B are heat maps 1700-A and 1700-B, respectively, of example embodiments of comorbidity comparisons for cardiomyopathy hypertrophic disease LCC using shortest-path method between each module. In FIG. 17A, results of a shortest-path analysis for comorbid and unrelated modules with pre-trained Geneformer are shown. In FIG. 17B, results of a shortest-path analysis for comorbid and unrelated modules with fine-tuned cardiomyopathy Geneformer are shown.

### Dataset Size Impact of Bulk Results

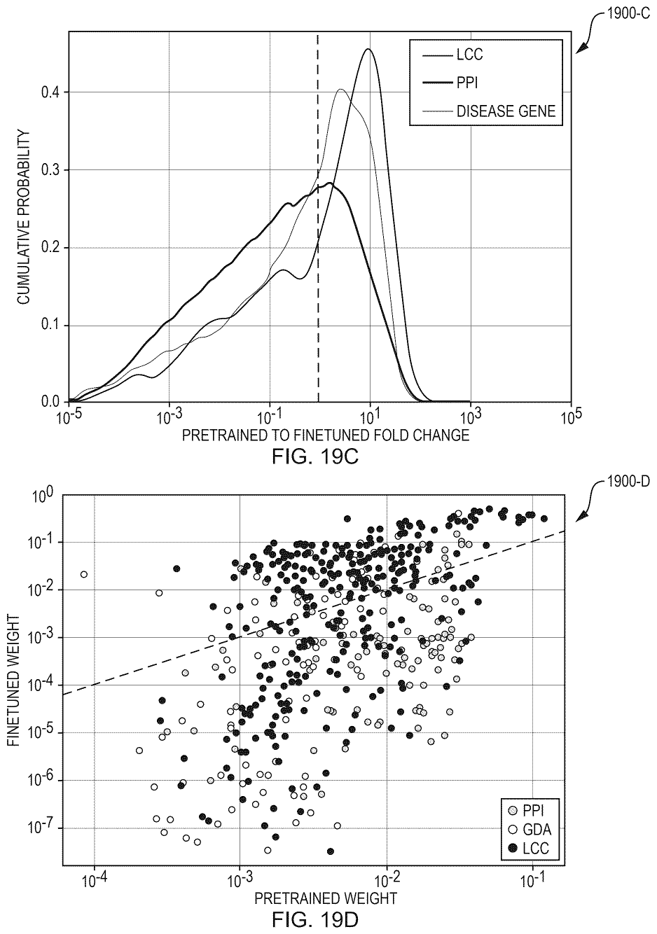

In certain datasets, Geneformer handily outperforms or matches the performance of other deterministic models, but in other datasets the performance of Geneformer lags behind. Understanding the data-intensive nature of transformer models, a study of the effect of dataset size on Geneformer model performance was performed and results shown in FIGS. 18A-C. As shown in FIG. 18A, the size of the label-equalized dataset has a strong correlation with the overall Geneformer model performance. In addition, in FIGS. 18B and 18C, it can be observed that for both the single-cell cardiomyopathy dataset and the bulk-RNA lung cancer dataset, as down-sampling continues, the model performance rapidly decreases, eventually saturating at a low level of classification effectiveness.

FIGS. 18A-C are plots 1800-A, 1800-B, and 1800-C, respectively, of example embodiments of a relationship between dataset size and model performance for bulk-RNA and single-cell RNA data with Geneformer. In FIG. 18A, the plot 1800-A is a box-plot with trendline of dataset size against GF model performance for small-cell lung carcinoma, lung carcinoma, breast cancer, and rheumatoid arthritis. In FIG. 18B, the plot 1800-B shows a distribution analysis of 3 GF runs on the single-cell cardiomyopathy hypertrophic dataset sub-sampled down to the given amount of samples. In FIG. 18C, the plot 1800-C shown a distribution analysis of 3 GF runs on the bulk-RNA rheumatoid arthritis dataset sub-sampled down to the given amount of samples.

### Non-Coding Interactome Results

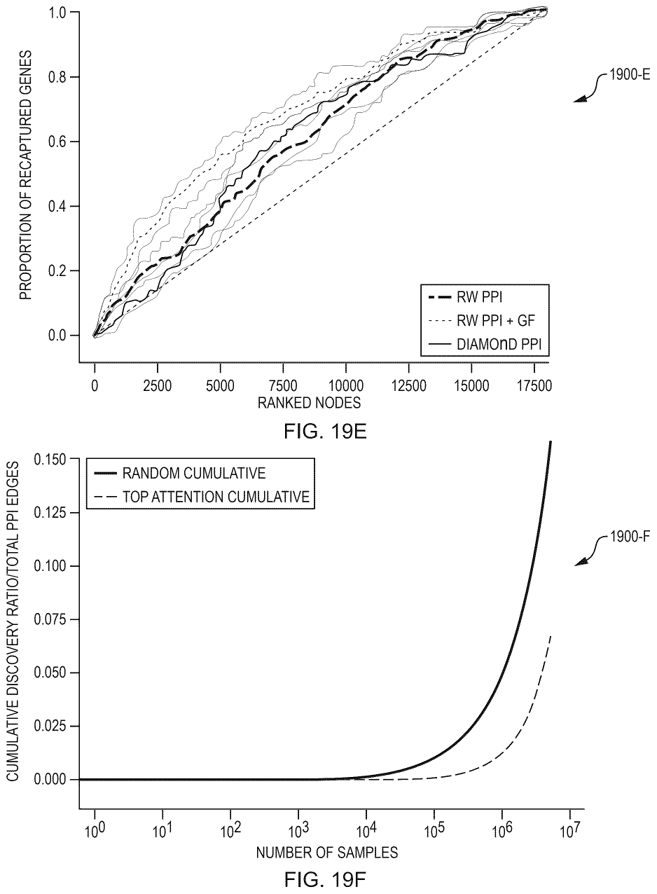

An investigation was performed to determine if the network-based effects of mapping Geneformer attention weights holds for non-coding interactomes compared to the coding interactome used herein. The investigation made use of a non-coding interactome (Barabási N. Gulbahce, and J. Loscalzo, Nat Rev Genet 12, 56 (2011) that has been extensively validated, and ran several of the same analyses with the pre-trained Geneformer model and fine-tuned cardiomyopathy model. It was observed that the pre-trained model still can recognize the PPI distribution, especially on the upper tail of the distribution (FIGS. 19A, 19B, and 19F). It was further observed that the attention weight distribution and scatter plot display the same preferential amplification of the LCC, with fold change values even larger than the coding interactome (with an average fold change of 6.09 for the LCC compared to 4.19 for the coding interactome) (FIGS. 19C and 19D). Finally, it was observed that the LCCs of cardiomyopathy are preferentially amplified compared to the pre-trained model (FIG. 19D), and that the NCI fine-tuned weights can be used effectively for disease module recapture (FIG. 19E)

FIGS. 19A-F are graphs (1900-A, 1900-B, 1900-C, 1900-D, 1900-E, and 1900-F) of example embodiments of results of a network-based analysis of non-coding human interactome (NCI) Geneformer (GF) attention weights. In FIG. 19A, attention weight probability distributions for pre-trained Geneformer are mapped to NCI. In FIG. 19B, ROC curves of mapping attention weights to true and false NCI edges are plotted. In FIG. 19C, fold change between pre-trained GF and fine-tuned cardiomyopathy GF attention weights for NCI edges for LCC edges, connected GDA disease edges, and PPI edges are plotted. In FIG. 19D, the graph 1900-D is a scatter plot of cardiomyopathy hypertrophic attention weights mapped to LCC edges and a random balanced subsample of connected GDA edges and PPI edges from NCI. FIG. 19E includes plots Capture/Recapture disease module discovery with half of the original module removed for random walk weighted with fine-tuned GF edges, unweighted random walk, and DIAMOND clustering on NCI. FIG. 19F shows cumulative proportion of PPI edges captured by 10 million top ranked attention weights and randomly sampled weights for NCI.

FIG. 20 is a block diagram of an example of an internal structure of a computer 2000 in which various embodiments of the present disclosure may be implemented. The computer 2000 contains a system bus 2018, where a bus is a set of hardware lines used for data transfer among the components of a computer or digital processing system. The system bus 2018 is essentially a shared conduit that connects different elements of a computer system (e.g., processor, disk storage, memory, input/output ports, network ports, etc.) that enables the transfer of information between the elements. Coupled to the system bus 2018 is an I/O device interface 2003 for connecting various input and output devices (e.g., keyboard, mouse, display monitors, printers, speakers, microphone, etc.) to the computer 2000. A network interface 2007 allows the computer 2000 to connect to various other devices attached to a network (e.g., global computer network, wide area network, local area network, etc.). Memory 2009 provides volatile or non-volatile storage for computer software instructions 2011 and data 2017 that may be used to implement embodiments (e.g., method 300) of the present disclosure, where the volatile and non-volatile memories are examples of non-transitory media. Disk storage 2074 also provides non-volatile storage for the computer software instructions 2011 and data 2017 that may be used to implement embodiments (e.g., method 300) of the present disclosure. A central processor unit 2072 is also coupled to the system bus 2018 and provides for the execution of computer instructions.

Example embodiments disclosed herein may be configured using a computer program product. Further example embodiments may include a non-transitory computer-readable medium that contains instructions that may be executed by a processor, and, when loaded and executed, cause the processor to complete methods described herein.

In addition, the elements described herein may be combined or divided in any manner in software, hardware, or firmware. If implemented in software, the software may be written in any language that can support the example embodiments disclosed herein. The software may be stored in any form of computer readable medium, such as random-access memory (RAM), read-only memory (ROM), compact disk read-only memory (CD-ROM), and so forth.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A computer-based system for network medicine, the network medicine employing representations of interactions of biological components, the computer-based system comprising:
at least one processor and a memory, the memory having encoded thereon a sequence of instructions which, when loaded and executed by the at least one processor, causes the computer-based system to:
employ a machine-readable representation of a transformer weighted protein-protein interactome (PPI) network to perform a computer-informed network medicine task, the machine-readable representation of the transformer weighted PPI network including machine-readable links weighted based on biological context information extracted from a transformer-based model, the machine-readable links interposed between machine-readable representations of proteins in the transformer weighted PPI network, the biological context information extracted from the transformer-based model based on machine-readable genetic data; and
output a result of the computer-informed network medicine task performed.

2. The computer-based system of claim 1, wherein the computer-informed network medicine task includes at least one of: disease gene discovery and drug repurposing.

3. The computer-based system of claim 1, wherein the machine-readable genetic data represents genetic data of cells of an individual with a disease or genetic data of cells of a plurality individuals associated with the disease.

4. The computer-based system of claim 1, wherein the computer-informed network medicine task performed includes predicting, from an input set of candidate drugs, which candidate drugs of the input set are useful to treat a disease, wherein the result output is a prioritized list of candidate drugs from the input set of candidate drugs predicted to treat the disease, wherein a subset of the input set of candidate drugs is known to treat the disease, and wherein a total number of candidate drugs in the prioritized list of candidate drugs is reduced relative to a total number of candidate drugs in the input set.

5. The computer-based system of claim 1, wherein the computer-informed network medicine task performed includes predicting from an input set of candidate drugs known to treat a disease and a machine-readable genetic profile of an individual with the disease, which candidate drugs of the input set are useful to treat the disease in the individual, wherein the result output is a prioritized list of candidate drugs from the input set predicted to treat the disease, and wherein a location within the prioritized list represents probability of success in treating the disease.

6. The computer-based system of claim 1, wherein the computer-informed network medicine task performed includes predicting from an input set of candidate drugs and a machine-readable genetic profile of an individual with a disease, which candidate drugs of the input set are useful to treat the disease in the individual, and wherein the result output is a prioritized list of candidate drugs from the input set predicted to treat the disease.

7. The computer-based system of claim 1, wherein the computer-informed network medicine task performed includes discovering new genes associated with a disease based on known genes associated with the disease and wherein the result output is a prioritized list of new genes determined to be associated with the disease.

8. The computer-based system of claim 1, wherein the computer-informed network medicine task performed includes determining which drugs of an input set of candidate drugs with respective disease targets can be used to treat a disease different from the respective disease targets and wherein the result output is a prioritized list of the input set.

9. The computer-based system of claim 1, wherein the sequence of instructions, when loaded and executed by the at least one processor, further causes the computer-based system to transform a machine-readable representation of an unweighted PPI into the transformer weighted PPI network by determining weights based on the biological context information extracted from the transformer-based model and assigning the weights determined to machine-readable links interposed between machine-readable representations of proteins in the unweighted PPI network.

10. The computer-based system of claim 1, wherein the biological context information extracted from the transformer-based model includes machine-readable vectors of different layers of the transformer-based model that represent respective expression levels of genes in cells represented in the machine-readable genetic data, wherein the sequence of instructions, when loaded and executed by the at least one processor, further causes the computer-based system to compute angles between the machine-readable vectors of the different layers, and wherein the machine-readable links are weighted based on the angles computed.

11. The computer-based system of claim 10, wherein the sequence of instructions, when loaded and executed by the at least one processor, further causes the computer-based system to aggregate the angles computed, compute an average value of the angle computed and aggregated, and assign weights to the machine-readable links based on the average value computed.

12. The computer-based system of claim 1, wherein the biological context information extracted from the transformer-based model includes attention weights extracted from the transformer-based model and wherein the sequence of instructions, when loaded and executed by the at least one processor, further causes the computer-based system to assign weights to the machine-readable links based on the attention weights extracted.

13. A computer-implemented method for network medicine, the network medicine employing representations of interactions of biological components, the computer-implemented method comprising:
employing a machine-readable representation of a transformer weighted protein-protein interactome (PPI) network to perform a computer-informed network medicine task, the machine-readable representation of the transformer weighted PPI network including machine-readable links weighted based on biological context information extracted from a transformer-based model, the machine-readable links interposed between machine-readable representations of proteins in the transformer weighted PPI network, the biological context information extracted from the transformer-based model based on machine-readable genetic data; and
outputting a result of the computer-informed network medicine task performed.

14. The computer-implemented method of claim 13, wherein the computer-informed network medicine task includes at least one of: disease gene discovery and drug repurposing and wherein the machine-readable genetic data represents genetic data of cells of an individual with a disease or genetic data of cells of a plurality individuals associated with the disease.

15. The computer-implemented method of claim 13, wherein the computer-informed network medicine task performed includes predicting, from an input set of candidate drugs, which candidate drugs of the input set are useful to treat a disease, wherein the result output is a prioritized list of candidate drugs from the input set of candidate drugs predicted to treat the disease, wherein a subset of the input set of candidate drugs is known to treat the disease, and wherein a total number of candidate drugs in the prioritized list of candidate drugs is reduced relative to a total number of candidate drugs in the input set.

16. The computer-implemented method of claim 13, wherein the computer-informed network medicine task performed includes at least one of:
(i) predicting from a first input set of candidate drugs known to treat a first disease and a first machine-readable genetic profile of a first individual with the first disease, which candidate drugs of the first input set are useful to treat the first disease in the first individual, wherein the result output is a first prioritized list of candidate drugs from the first input set predicted to treat the first disease, and wherein a location within the first prioritized list represents probability of success in treating the first disease;
(ii) predicting from a second input set of candidate drugs and a second machine-readable genetic profile of a second individual with a second disease, which candidate drugs of the second input set are useful to treat the second disease in the second individual, wherein the result output is a second prioritized list of candidate drugs from the second input set predicted to treat the second disease, and wherein a location within the second prioritized list represents probability of success in treating the second disease;
iii) discovering new genes associated with a third disease based on known genes associated with the third disease, wherein the result output is a third prioritized list of new genes determined to be associated with the third disease, and wherein a location within the third prioritized list represents probability of success in treating the third disease; and
iv) determining which drugs of a third input set of candidate drugs with respective disease targets can be used to treat a fourth disease different from the respective disease targets, wherein the result output is a fourth prioritized list of the third input set, and wherein a location within the fourth prioritized list represents probability of success in treating the fourth disease.

17. The computer-implemented method of claim 13, further comprising transforming a machine-readable representation of an unweighted PPI into the transformer weighted PPI network by determining weights based on the biological context information produced by the transformer-based model and assigning the weights determined to machine-readable links interposed between machine-readable representations of proteins in the unweighted PPI network.

18. The computer-implemented method of claim 13, wherein the biological context information extracted from the transformer-based model includes machine-readable vectors of different layers of the transformer-based model that represent respective expression levels of genes in cells represented in the machine-readable genetic data, wherein the computer-implemented method further comprises:
computing angles between the machine-readable vectors of the different layers, wherein the machine-readable links are weighted based on the angles computed;
aggregating the angles computed;
computing an average value of the angle computed and aggregated; and
assigning weights to the machine-readable links based on the average value computed.

19. The computer-based system of claim 13, wherein the biological context information extracted from the transformer-based model includes attention weights extracted from the transformer-based model and wherein the computer-implemented method further comprises assigning weights to the machine-readable links based on the attention weights extracted.

20. A non-transitory computer-readable medium for network medicine, the network medicine employing representations of interactions of biological components, the non-transitory computer-readable medium having encoded thereon a sequence of instructions which, when loaded and executed by at least one processor, causes the at least one processor to:
employ a machine-readable representation of a transformer weighted protein-protein interactome (PPI) network to perform a computer-informed network medicine task, the machine-readable representation of the transformer weighted PPI network including machine-readable links weighted based on biological context information extracted from a transformer-based model, the machine-readable links interposed between machine-readable representations of proteins in the transformer weighted PPI network, the biological context information extracted from the transformer-based model based on machine-readable genetic data; and
output a result of the computer-informed network medicine task performed.
